(19) United States
(12) Patent Application Publication
(10) Pub. No.: US 2006/0167223 A1
(43) Pub. Date: Jul. 27, 2006

(54) Novel ampiphilic fluorocarbon molecular vectors for biomedical and medical use

(76) Inventors: Bernard Pucci, Molleges (FR); Ange Polidori, Avignon (FR); Christiane Contino-Pepin, Althen Des Paluds (FR); Gregory Durand, Villeneuve Les Avignon (FR); Sylvain Jasseron, Rochefort Du Gard (FR); Sandrine Perino, Domazan (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/533,983

(22) PCT Filed: Nov. 7, 2003
(86) PCT No.: PCT/FR03/03336

§ 371 Date: Sep. 29, 2005

(30) Foreign Application Priority Data
Nov. 8, 2002 (FR) 0214077

(51) Int. Cl. C07K 9/00

(52) U.S. Cl. 530/322; 536/53

(57) ABSTRACT

Novel ampiphilic fluorocarbon molecules applicable as vectors for active ingredients, active molecules comprising such a vector and the use thereof in the pharmaceutical field, particularly for the production of medicaments.

The invention relates to novel molecules that can be used as vectors for active principles, to active molecules comprising such a vector and to the use thereof in the pharmaceutical field, in particular for preparing medicinal products.

Current research on the delivery of active principles tends to notably improve not only the patient's comfort by favoring the least traumatic routes of administration, but also the overall effectiveness of a medicinal product by conferring on it a cellular affinity that it generally lacks and that is often responsible for adverse side effects. Added to the conventional concept of therapeutic activity is therefore that of the specificity of action, which in fact modulates the bioavailability of the substrate.

In practice, for example in a sensitive field such as that of anticancer chemotherapy, the progress observed over the last few years (novel drugs, novel methods of administration, chemopredictivity in vitro or in vivo, integration into novel therapeutic schemes, etc.) makes it possible to think that the quality of chemotherapy will continue to progress if, from now on, it can be equipped with novel tools based on novel concepts for transport and cellular targeting of the anticancer agents. In order to increase the effectiveness and the effective dose of active principle which will be conveyed to the cancerous site, it in fact appears to be necessary to give these medicinal products a real specificity and to reduce their side effects.

Now, the chemical structure of a medicinal product conditions both its physicochemical properties and its biological activity, and in particular its affinity for membrane receptors. Modulating this outcome through modifications of the molecular structure risks altering the pharmaceutical properties. One is therefore led to treat the product from a “galenic” point of view, i.e. to take an interest in the pharmaceutical form used for its administration, or better still to encapsulate it in host structures or graft it onto molecules capable of providing this vectorization.

The vectorization of active principles must take into account several parameters in order to have a chance of success:

As has just been specified, the active principle should, as much as is possible, be isolated from the physiological medium in order to prevent any interaction which is harmful to the medicinal product or to the organism.

The carrier should not impair, or better still should improve, the bioavailability of the medicinal product. In other words, the therapeutic agent should be released within the target molecule (preferably at the intracytoplasmic level, in certain cases at the intranuclear level) and should conserve therein its entire activity.

However, this vision of the medicinal product, an agent specific for a receptor, that is capable of causing a cellular response and, as a result, of correcting a deficiency is far from being general. While it is correct for hormone or analgesic-type active principles, it does not apply at all to other fields, for instance anticancer treatments. Such substrates were not designed in this way: they do not have any cell recognition specificity. Their aim is to inhibit cell multiplication. They are generally antimitotic agents and, as a result of this, they can act on the DNA of all cells, whether they are cancerous or normal and can thus create certain bothersome conditions, the most common manifestation of which is hematopoietic tissue aplasia, to which is subsequently added immuno-inhibition and digestive problems. Since the establishment of chemotherapy, the active principles developed have been increasingly powerful, but unfortunately do not distinguish between cancer cells and normal cells. It is thus regrettable that effectiveness and selectivity in terms of cancer treatment cannot be conjugated on the same active principle.

Given these various considerations and observations, various vector models have been proposed, essentially of macromolecular type (synthetic or natural polymers) and supramolecular type (liposomes). Among all these vector models, mention may in particular be made of the development of small amphiphilic polymers, called telomers, capable of modulating the hydrophilicity-lipophilicity balance of the active principle (and therefore its intrinsic physicochemical properties), but also of promoting its intracellular penetration and of providing it with cellular targeting by means of suitably chosen recognition agents.

“Synthesis of new cotelomers derived from tris(hydroxymethyl)aminomethane bearing arabinofuran-osylcytosine moieties. Preliminary results on their in vitro and in vivo antitumoral activities” C. Contino, J. C. Maurizis, M. Ollier, M. Rapp, J. M. Lacombe, B. Pucci. Eur. J. Med. Chem., (1998), 33, 809-816.

“Synthesis and preliminary biological assessments of a new class of amphiphilic telomers bearing 5-fluorouracil moieties” C. Contino, J. C. Maurizis and B. Pucci. Macromol. Chem., (1999), 200, 1351-1355.

“A new strategy in biomedical and medical field: the synthesis and applications of telomeric structures”. P. Barthelemy, A. Polidori, B. Pucci. Transworld Research Network, Recent developments in organic chemistry, Trivandrum, (1999), 3, 117-140.

“Synthesis and Preliminary biological assessments of RGD bearing biocompatible telomers. Sylvain Jasseron, Christiane Contino-Pépin, Jean Claude Maurizis, Maryse Rapp, Bernard Pucci. Bio. Med. Chem. Letters, (2002), 12, 1067-1070.

“Synthesis and preliminary biological assessments of a new class of amphiphilic telomers bearing 5-fluorouracil moieties” C. Contino, J. C. Maurizis and B. Pucci. Macromol. Chem., (1999), 200, 1351-1355.

“Amphiphilic telomers: a new kind of antimitotic drugs macromolecular carriers.” Christiane Contino-Pépin, Jean-Claude Maurizis, Bernard Pucci. Curr. Med. Chem.-Anti-Cancer Agents, (2002), 2, 645-665.

The results acquired in the course of these studies have made it possible to demonstrate various major points:

Control of the hydrophilicity-lipophilicity balance of the substrate promotes its transmembrane passage (“Uptake and subcellular distribution of a new fluorinated telomeric carrier: study on cultivated B16 melanoma and skin rat fibroblastic cells”. F. Chehade, J. C. Maurizis, B. Pucci, A. A. Pavia, M. Ollier, A. Veyre, F. Escaig, C. Jeanguillaume, R. Dennebouy, G. Slodzian, E. Hindie, Cellular and Molecular Biology, (1996), 42, 335-342) without, however, introducing a detergent and therefore toxic nature (“Efficiency of new non ionic telomeric surfactants towards the solubilization of subcellular fraction proteins” B. Pucci, J. C. Maurizis and A. A. Pavia, BioOrg. Med. Chem Lett. (1993), 3, 161-164).

These amphiphilic polymers make it possible to provide the overall molecule and therefore the active principle with effective cell targeting (“Cell targeting by glycosidic telomers—Recognition ability of galactosylated telomers by the yeast Kluyveromyces Bulgaricus” J. Coulon, R. Bonaly, B. Pucci, A. Polidori, P. Barthelemy, C. Contino, Bioconjugate Chem. (1998), 9, 152-159. “Permeability of yeast cell enveloppe to fluorescent galactosylated telomers derived from THAM”. C. Contino, M. Briot, J. Coulon, A. Polidori, R. Bonaly and B. Pucci. Bioconjugate Chem., (2000), 11, 461-468. “Synthesis and Preliminary biological assessments of RGD bearing biocompatible telomers”. Sylvain Jasseron, Christiane Contino-Pépin, Jean-Claude Maurizis, Maryse Rapp, Bernard Pucci. Bio. Med. Chem. Letters, (2002), 12, 1067-1070).

Active principles grafted onto the vector by means of a suitable peptide spacer arm (that can be hydrolyzed by cytoplasmic enzymes) are released at the intracellular level after the vector has passed through the cell membrane (“Synthesis and Preliminary biological assessments of RGD bearing biocompatible telomers”. Sylvain Jasseron, Christiane Contino-Pépin, Jean Claude Maurizis, Maryse Rapp, Bernard Pucci. Bio. Med. Chem. Letters, 12, 1067-1070).

This method of vectorization makes it possible to very significantly increase the effectiveness of the anticancer agent since it inhibits the proliferation of metastases, slows down tumor growth, and prolongs the lifetime of treated mice by a factor of more than 3 compared with control mice.

Despite the obvious advantage provided by these telomers which have been described in document WO 92/02560, one of the major problems with which such vectors are confronted and which could disrupt the commercialization and the use thereof is their polydispersity, i.e. their lack of well-defined mass and structure.

Thus, the Applicant set itself the objective of designing and preparing molecules capable of being vectors, for an active principle, which have a well-defined structure, the preparation of which is easy, and which facilitate the delivery of the active principle to its target.

A subject of the invention is therefore the molecules corresponding to formula (I) below:

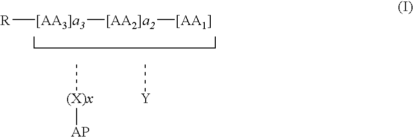

in which:

AP represents the active principle capable of acting on a biological target and the delivery of which to its biological target it is desired to promote;

x represents an integer chosen from 0 and 1;

X represents a peptide chain comprising from 1 to 5 amino acids;

AA₁, AA₂ and AA₃, which may be identical or different, each represent an amino acid;

a₂ and a₃, which may be identical or different, each represent an integer chosen from 0 and 1;

R represents a group chosen from a targeting agent and a solubilizing agent. For the purpose of the present invention, the term “targeting agent” is intended to mean: a molecule that promotes the delivery of the entire molecule of formula (I) to its target or any molecule capable of being recognized by the target of the active principle AP. The term “solubilizing agent” is intended to mean an agent for modulating the HLB balance of the molecule of formula (I), in particular a hydrophilic agent. Among the targeting agents that can be used in the present invention, mention may be made of monosaccharides, aminated derivatives of sugars, polysaccharides, natural or synthetic hormones, peptides, antibodies and, generally, any molecule capable of being recognized by the target of the active principle AP. Among the solubilizing agents that can be used in the present invention, mention may in particular be made of polyols, polyethers, peptides and polysaccharides.

Y represents a fluorinated C₄-C₁₂ hydrocarbon-based chain containing a group 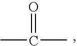
—NH—, —O—CO—NH—, S or O that allows its attachment, indicated by the dashes ---, either to one of the ends of the peptide chain [AA₃]ₐ₃-[AA₂]ₐ₂-[AA₁], or to the side chain of one of the amino acids AA₁, AA₂ or AA₃;

The dashes --- between AP-(X)ₓ and the chain [AA₃]ₐ₃-[AA₂]ₐ₂-[AA₁] indicate that the linkage of AP-(X)ₓ with the rest of the molecule occurs via the side chain of one of the amino acids AA₁, AA₂ or AA₃ or, optionally, at the end of the peptide chain.

More particularly, the active principle is chosen from all organic molecules that have a recognized biological activity and that are capable of being attached to an amino acid by means of a linkage that can be chosen from the functions —O—CO—, —CO—NH—, —NH—CO—NH—, —NH—CO—O—, O—CO—O—, —O—, —S—,

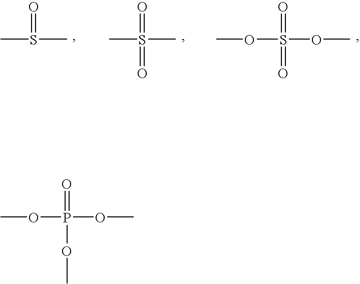

Among these active principles, mention may in particular be made of those that have anticancer, anti-inflammatory, antiseptic, analgesic, neuroleptic or antifungal activity, and molecules that have free-radical scavenger activity.

In general, the active principle may consist of a linear, branched or cyclic molecule containing from 1 to 30 carbon atoms, one or more unsaturations, in particular one or more aromatic rings, and one or more functions chosen from:

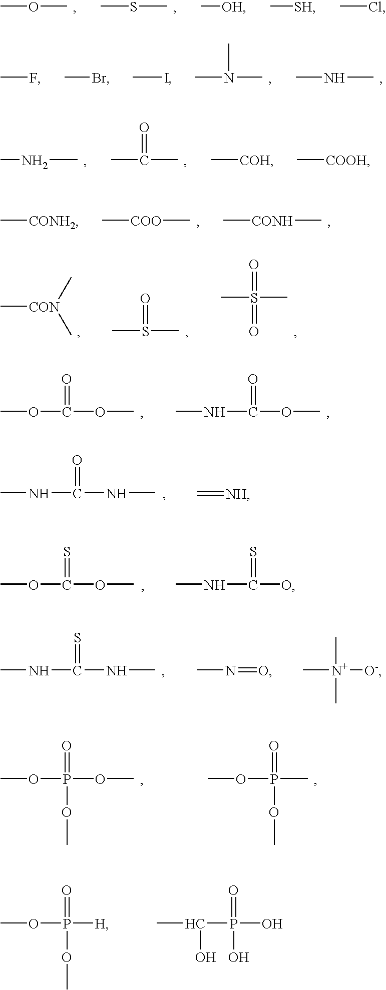

The active principle AP is attached, by means of a linkage the nature of which was disclosed above, either to the side chain of one of the amino acids AA₁, AA₂ or AA₃, or to the end of the peptide chain, optionally by means of a peptide chain X (in the case where x=1).

The linkage with one of the two groups Y and —(X)x-AP takes place on the side chain of one of the amino acids AA₁, AA₂ or AA₃. The amino acid attached to AP—(X)ₓ— or to Y via its side chain is chosen from those containing an acid, amide, amine, thiol or alcohol function on their side chain. Among these, mention may in particular be made of lysine, arginine, ornithine, aspartic acid, glutamic acid, asparagine, glutamine, serine, tyrosine or cysteine. Preferably, the amino acid attached via its side chain to AP—(X)ₓ— or to Y is chosen from: aspartic acid or lysine.

The spacer arm X, when it is present, consists of a peptide chain involved, at one end, in a linkage with the side chain or the end of one of the amino acids AA₁, AA₂ or AA₃ and, at the other end, in a linkage with the active principle AP.

This spacer arm comprises 1 to 5 amino acids, preferably 1 to 3 amino acids.

The spacer arm X and/or the peptide chain [AA₃]ₐ₃-[AA₂]ₐ₂-[AA₁] can be chosen for their affinity for the target of the active principle AP. They may also comprise or consist of tyrosine residues that make it possible to follow the molecule of formula (I) in vivo, after labeling with ¹²⁵I.

R is chosen according to the cell target; it may be saccharide in nature (targeting of specific membrane lectins that are in specific tissues and that selectively recognize either galactose—in the case of the liver, of bones, of certain cancerous tumors—, or mannose—in the case of macrophages, of the heart—, or sialic acid—in the case of erythrocytes—, etc.), hormonal in nature (such as steroids), or synthetic in nature such as imatinib mesylate (ST571, Gleevek®) for targeting kinases, specific antibodies, in particular peptides. R can be chosen from any substrates for which prior research has demonstrated the recognition specificity. When R is a monosaccharide or polysaccharide or a hydrophilic peptide, it may, in addition, provide the molecule with the water-solubility necessary for its IV or IP administration.

When R is a peptide chain, R advantageously contains from 3 to 15 amino acids, even more advantageously from 3 to 10 amino acids. It may also contain one or more tyrosine residues that make it possible to follow the molecule of formula (I) in vivo, after labeling with ¹²⁵I.

The amino acids constituting the spacer arm X, just like those constituting the chain [AA₃]ₐ₃-[AA₂]ₐ₂-[AA₁] or the group R are chosen from natural amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine, or non-natural amino acids such as hydroxyl-proline, norleucine, ornithine, citruline or cyclohexylalanine.

The use of Ω-amino acids such as 3-aminopropionic acid and 4-aminobutyric acid can also be envisioned.

When R is a peptide, the peptide chain R may be an antibody fragment or epitope having a pronounced affinity for the AP's biological target.

Among the peptides that can be used in the present invention, mention may be made, for example, of: the RGD sequence, known for its affinity for αVβ3 integrins.

R can also be chosen from polyols or polyethers, in particular poly(ethylene oxide)s, so as to give the molecule of formula (I) a hydrophilic/lipophilic balance that promotes its solubility in water and its penetration into the cell as far as the target of the active principle AP.

When R consists of a polyol, said polyol advantageously consists of an alkyl chain comprising from 4 to 16 carbon atoms and from 4 to 16 hydroxyl groups.

When R consists of a poly(ethylene oxide) chain as solubilization unit, said chain advantageously comprises from 5 to 30 ethylene oxide units.

R can in particular be chosen from monosaccharides, aminated derivatives of sugars, and polysaccharides.

Among the monosaccharides that can be used in the present invention, mention may be made of: glucose, fructose, mannose, galactose and ribose. Among the aminated derivatives of sugars, mention may in particular be made of glucosamine. Among the polysaccharides that can be used in the present invention, mention may be made of lactose, cellobiose or maltose, lactobionamide and sucrose. Preferably, the polysaccharide chains used in the invention are bisaccharides.

The attachment of R to one of the ends of the chain [AA₃]ₐ₃-[AA₂]ₐ₂-[AA₁] takes place by means of a suitable linkage: ether, amide, carbamate, thioether, ester, urea, urethane, according to the functionality that can be grafted onto R.

The fluorinated hydrocarbon-based chain is preferably chosen from those corresponding to the formula A-Y′ in which A represents a group chosen from: 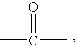
—NH—, —O—CO—NH—, S and O, and Y′ represents a molecule corresponding to the formula —(CH₂)ₜ—(CF₂)ᵣF, in which r and t represent two integers with: 12≧r+t≧4, such as, for example:
—(CF₂)₄F; —(CF₂)₅F; —(CF₂)₆F; —(CF₂)₇F; —(CF₂)₈F; —(CF₂)₉F; —(CF₂)₁₀F; —(CF₂)₁₁F; —(CF₂)₁₂F; —(CF₂)₁₃F; —(CF₂)₁₄F; —CH₂₋(CF₂)₃F; —CH₂—(CF₂)₄F; —CH₂—(CF₂)₅F; —CH₂—(CF₂)₆F; —CH₂—(CF₂)₇F; —CH₂—(CF₂)₈F; —CH₂—(CF₂)₉F; —CH₂—(CF₂)₁₀F; —CH₂—(CF₂)₁₁F; —CH₂—(CF₂)₁₂F; —CH₂—(CF₂)₁₃F; —(CH₂)₂—(CF₂)₂F; —(CH₂)₂—(CF₂)₃F; —(CH₂)₂—(CF₂)₄F; —(CH₂)₂—(CF₂)₅F; —(CH₂)₂—(CF₂)₆F; —(CH₂)₂—(CF₂)₇F; —(CH₂)₂—(CF₂)₈F; —(CH₂)₂—(CF₂)₉F; —(CH₂)₂—(CF₂)₁₀F; —(CH₂)₂—(CF₂)₁₁F; —(CH₂)₂—(CF₂)₁₂F; —(CH₂)₃—(CF₂)₁F; . . . —(CH₂)₁₁—(CF₂)F. Preferably, t≧2. Preferably, 12≧r≧4, even more preferably 10≧r≧6.

A subject of the invention is also any biologically active molecule containing a fragment of formula (II) 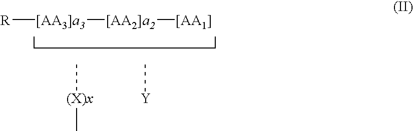
in which R, AA₁, AA₂, AA₃, a₂, a₃, Y, X and x have the same definition as in formula (I) above.

In fact, the invention provides a molecule fragment of formula (II) to which it is possible to attach, by means of a suitable linkage, an active principle of any nature, as disclosed above, so as to promote the penetration of this active principle into the human or animal organism and so as to allow this active principle to reach its biological target.

Specifically, the amphiphilic nature of the molecule promotes the passing through membranes, and the optional presence of an agent for specific recognition of the target with which the active principle is associated promotes its delivery to this target.

A subject of the invention is therefore also the use of a molecule fragment of formula (II) as defined above, for promoting the bioavailability of an active agent.

The preparation of the molecules of formula (I) is illustrated below by means of examples corresponding to several variants of the invention. More generally, use is made of methods of protection, deprotection and coupling of peptide synthesis, which methods are well known to those skilled in the art and are disclosed in particular in the work “The peptides” Gross and Meienhofer, 3 vols, Academic Press, New York, 1979-1981.

Among the molecules corresponding to formula (I), one of the particular subjects of the invention consists of the molecules corresponding to formula (Ia) below:

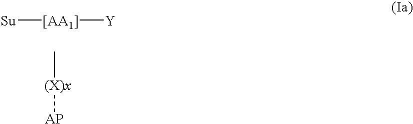

in which Su represents a variant of the group R, chosen from a monosaccharide, an aminated monosaccharide derivative, a polysaccharide, a polyol or, optionally, a polyether, as were defined above;

AA₁ represents an amino acid carrying an acid, amine, alcohol or thiol function on its side chain, by means of which it is attached either to (X)ₓ-AP or to Y; AA₁ is attached to Su and either to (X)ₓ-AP, or to Y, via its N- and C-terminal ends.

X, x, AP and Y have the same definition as in formula (I) above. Y is attached to the amino or acid end of AA₁ or, optionally, to its side chain.

Preferably, one or more of the conditions below are verified:

Su represents a monosaccharide or a polysaccharide;

X represents a spacer arm that is peptide in nature, containing at least one tyrosine residue; preferably, X represents tyrosine;

AA₁ represents an amino acid chosen from arginine and lysine;

Y represents a fluorinated C₆-C₁₂ hydrocarbon-based chain containing from 5 to 23 fluorine atoms, attached to the amino acid AA₁ via an —NH— function.

Two examples of compounds of formula (Ia) are illustrated below and in the examples:

### a) EXAMPLE 1

### Targeting of Angiogenic Sites

Angiogenesis is a natural biological process for creating new blood microvessels from pre-existing venules. It is a complex phenomenon that occurs normally in adults only under certain specific conditions such as wound healing, inflammation or the development of the corpus luteum during the menstrual cycle. Under normal conditions, the process of angiogenesis stops after an appropriate amount of time, indicating correct regulation of the stimulatory and inhibitory factors. Under certain pathological conditions, such as solid tumor growth, rheumatoid arthritis, psoriasis or diabetic retinopathy, angiogenesis develops in a clearly less controlled manner (“Antiangiogenic agents and their promising potential in combined therapy”, P. A. Burke, S. J. DeNardo, Crit. Rew. In Oncology/Hematology, (2001), 39, 155-171). More than 30 years ago, J. Folkman put forward the hypothesis that solid tumor growth was closely linked to the development of angiogenesis, and since then, a very large number of teams have shown an interest in this phenomenon and have tried to develop substrates capable of blocking the angiogenic process (“Tumor angiogenic therapeutic applications” J. Folkman Engl. J. Med. (1971), 285, 1182-1186 and “Tumor angiogenis past, present and the near future”. R. S. Kerbel Carcinogenesis (2000), 21, 505-521). Among the various structures tested, thalidomide, initially prescribed to pregnant women as a sedative and responsible for problems of teratogenesis, proved to be extremely advantageous for inhibiting vascular development. The idea that prevailed in the work carried out was to graft thalidomide onto the vector provided beforehand with a radioactive unit such as iodine 125-labeled tyrosine. The aim being sought in this example is to readily visualize the angiogenic sites in vivo, and therefore the solid tumors, and to block their development.

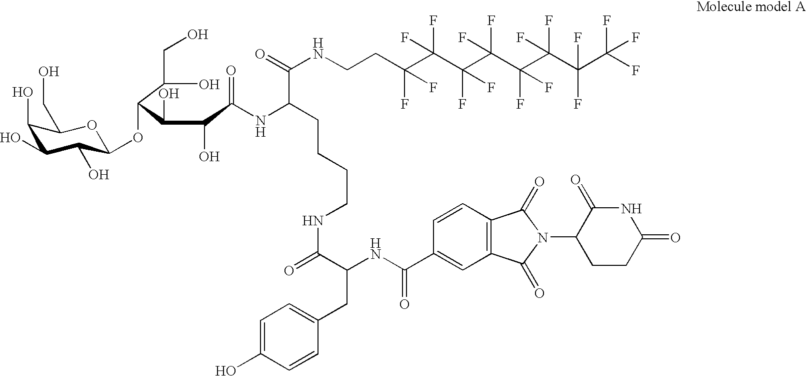

With this aim, a central lysine unit was provided with a fluorocarbon chain on the primary acid function, with a unit of lactose type capable of providing the molecule with the water-solubility necessary for its intravenous or intraperitoneal administration, and with tyrosine that is subsequently labeled with iodine 125 and onto which thalidomide, provided beforehand with a reactive acid function in the 3-position, is grafted.

According to a preferred variant of the invention, in the molecules corresponding to formula (Ia), the active principle is chosen from molecules capable of blocking the angiogenic process, in particular thalidomide.

### b) EXAMPLE 2

### Spin-Trap Vectorization

Mitochondrial cytopathies include a large variety of diseases, the common denominator of which is a deficiency in the mitochondrial respiratory chain. Due to the ubiquitous presence of mitochondria in the organism, this dysfunction can affect any organ. The effect can be isolated or, on the contrary, plurivisceral, in that case generally showing dominance in the neuromuscular system. No treatment currently exists for these diseases, which can be classified within the context of “orphan diseases”.

It is, however, now clear that, since mitochondria are, in the cell, the preferred site for the production of free radicals, deficiencies in the respiratory chain are very commonly associated with an overproduction of free radicals, the consequence of which is accelerated cell death in the affected tissues. Recent studies carried out at the Necker hospital in the team of Dr. P. Rustin (“Increased apoptosis in vivo in cells lacking mitochondrial DNA gene expression”, Wang J, Silva J P, Gustafsson C, Rustin P, Larsson N G. Proc Natl Acad Sci USA (2001) (in press)) have made it possible to show, on a series of human cells in culture, the importance of this production of free radicals of oxygen. These cell cultures represent all the types of deficiencies affecting the various complexes of the respiratory chain that are known in humans. They were characterized both from the point of view of the deficiency affecting the respiratory chain and from the point of view of the production of free radicals and of its consequences on cell survival. This collection of cells represents an irreplaceable tool for studying the effectiveness of any molecule whose target is the free-radical reactions associated with respiratory chain deficiencies.

The recent identification, in our team, of a “spin-trap” molecule capable of blocking cell death in cell models of apoptosis induced by free radicals produced by the respiratory chain has given us a basis for developing similar molecules exhibiting further enhanced effectiveness (“Superoxide-induced massive apoptosis in cultured skin fibroblasts harboring the Neurogenic Ataxia Retinitis Pigmentosa (NARP) mutation in the ATPase-6 gene of the mitochondrial DNA”. Geromel V, Kadhom N, Ceballos-Picot I, Ouari O, Polidori A, Munnich A, Rötig A, Rustin P. Hum Mol Genet (2001) (in press)). The aim pursued here was to refine and to simplify the structure of these substrates while at the same time conserving their biological activity in order to develop a synthetic process that can be readily adapted to the industrial phase. The tests were carried out on several cell models: cell cultures exhibiting a deficiency in the mitochondrial respiratory chain (fibroblasts in culture), neuron/muscle cell cocultures subjected to the action of free radicals and, finally, on cells extracted from skin having been subjected to a 3rd degree burn.

The purpose of the research undertaken was to have free-radical traps that can be used clinically for treating apoptotic phenomena, and more generally cell death phenomena attributable to the overproduction of free radicals. The extremely encouraging results obtained on these cell types fully justify the development of these amphiphilic vector models.

Molecule E, constructed on the previous molecule, was provided, in this particular case, with a well-known and effective spin-trap that contains a derivative of PBN.

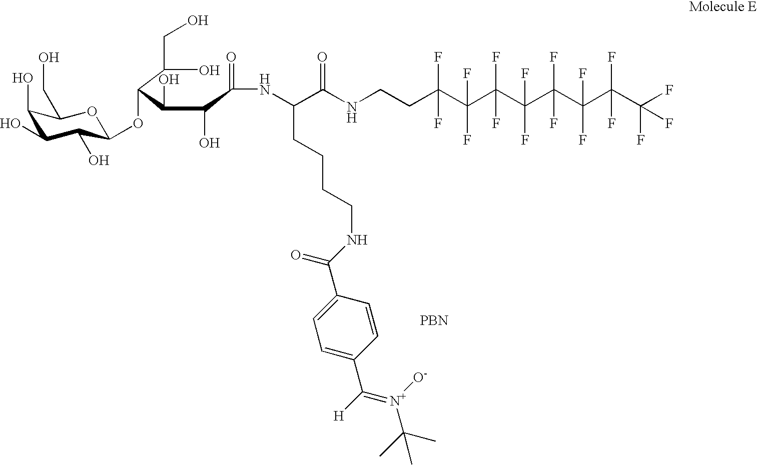

The first tests were carried out in vitro at the Necker hospital on fibroblasts derived from a biopsy of skin from a child exhibiting the NARP mutation. In a similar manner to the product TA1PBN (“Superoxide-induced massive apoptosis in cultured skin fibroblasts harboring the Neurogenic Ataxia Retinitis Pigmentosa (NARP) mutation in the ATPase-6 gene of the mitochondrial DNA”, Geromel V, Kadhom N, Ceballos-Picot I, Ouari O, Polidori A, Munnich A, Rötig A, Rustin P. Hum Mol Genet (2001) (in press) and “Synthesis of a glycolipidic amphiphile nitrone as a new spin trap for biological applications” O. Ouari, A. Polidori, F. Chalier, P. Tordo, B. Pucci. J. Org. Chem., (1994), 64, 3554-3556) previously tested, molecule E exhibits a capacity for cell protection and inhibits the apoptotic process. No toxicity was measured, with respect to this type of product, on any of the cells placed in culture.

These results validate once again the advantage of such a vectorization concept and clearly show its potentialities in fields of application that are entirely different.

According to another preferred variant of the invention, in the molecules corresponding to formula (Ia), the active principle is chosen from free-radical scavengers, in particular N-benzylidene-tert-butylamine oxide derivatives.

Among the molecules corresponding to formula (II), another particular subject of the invention consists of the molecules corresponding to formula (Ib):
Pep-[AA₁]-Y (Ib)

in which Y and AA₁ have the same definition as in formula (I) above, in particular as in formula (Ia), and Pep, which is a variant of R, represents a peptide chain containing from 2 to 10, preferably from 4 to 6, amino acids. Advantageously, Pep or AA₁ contains at least one tyrosine unit.

Advantageously, Pep is chosen for its affinity for a given biological target; in particular, this peptide chain can contain an RGD (arginine-glycine-aspartic acid) sequence that is known to be recognized by αVβ3 integrins.

Another subject of the invention consists of the molecules corresponding to formula (Ic):

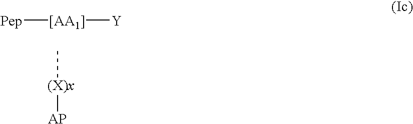

in which x, X, AP, AA₁ and Y have the same definition as in formula (I) above; in particular, the molecules in which x, X, AP, AA₁ and Y have the same definition as in formula (Ia) above; Pep has the same definition as in formula (Ib) above.

Preferably, one or more of the conditions below are verified:

Pep is a peptide recognized by αVβ3 integrins and AP is an antimitotic agent;

X, Pep or AA₁ contains at least one tyrosine residue;

X represents a chain of 1 to 3 amino acids.

An example of compounds of formulae (Ib) and (Ic) is illustrated below and in the experimental section:

### c) EXAMPLE 3

### Anticancer Therapy

At the current time, no technique has made it possible to demonstrate a criterion for effective differentiation, and therefore for appropriate targeting, of tumor cells and normal cells. As has been seen, however, the growth of a cancerous tumor is closely linked to its rate of vascularization and therefore to the phenomenon of angiogenesis that accompanies it. These observations had the scientific community to develop substrates, as has been seen, capable of inhibiting angiogenesis and therefore of blocking, by this means, the development of tumors. It is in fact now commonly accepted that membrane proteins carried by the angiogenic cells, and called integrins, participate actively in the process of proliferation of these cells. More particularly, αVβ3 integrins recognize a specific peptide sequence, the RGD (arginine-glycine-aspartic acid) sequence. The grafting of this unit onto the proposed vector should therefore provide it with an ability to specifically target angiogenic sites and therefore, ultimately, tumor sites. The addition, to this same vector, of an antimitotic agent should allow the selective destruction of the cancerous cells. In this perspective, molecule B was initially prepared to verify the innocuousness of the vector and to determine its specificity. In order to give the targeting agent a greater degree of freedom, said targeting agent was grafted onto the central lysine by means of a hydrophobic amino acid, serine. The molecule is water-soluble and is amphiphilic in nature.

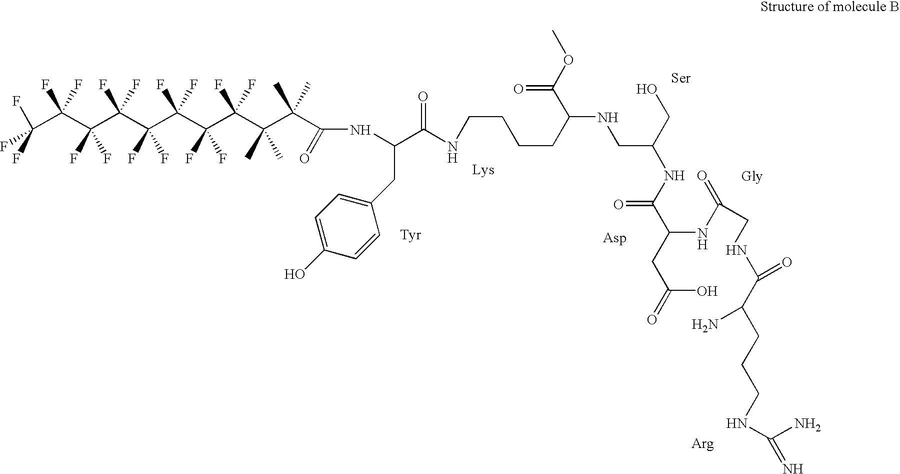

Given these positive results, further vectors carrying the RGD peptide sequence and an antimitotic agent such as melphalan (molecule D) or Ara-C (molecules C and F) were then synthesized and are in the process of being analyzed for their anticancer activity.

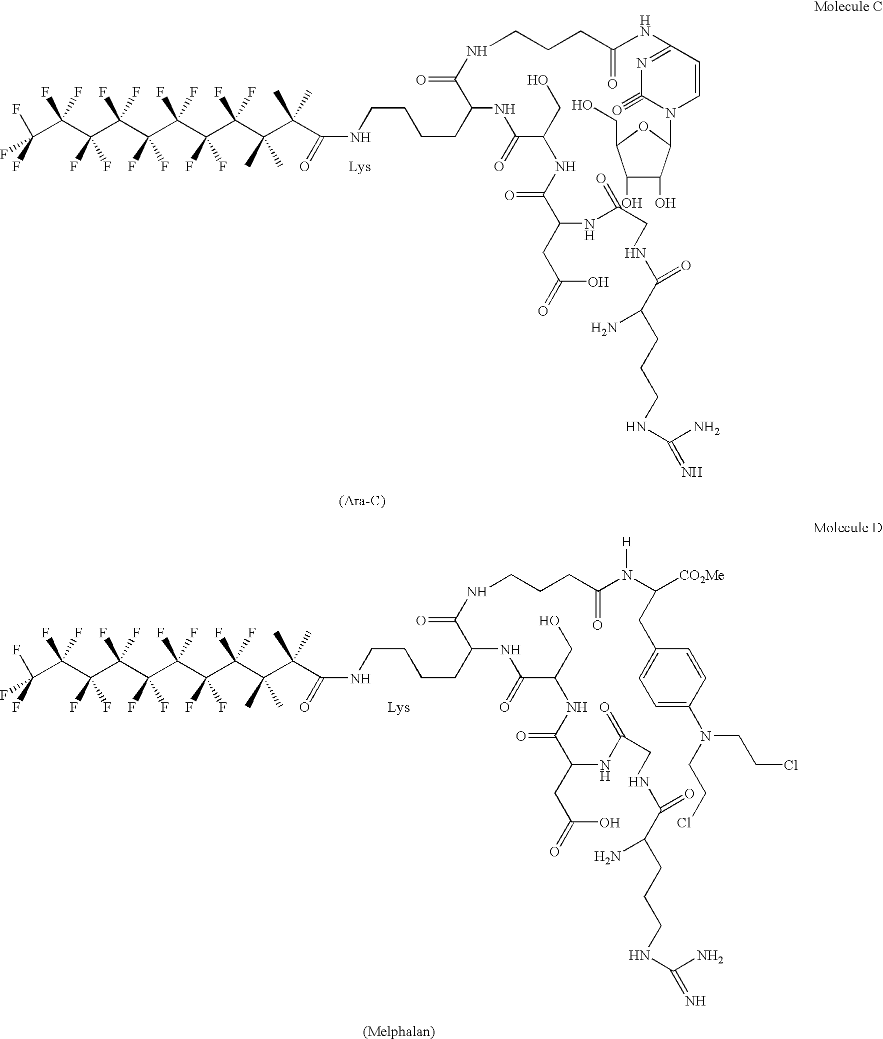

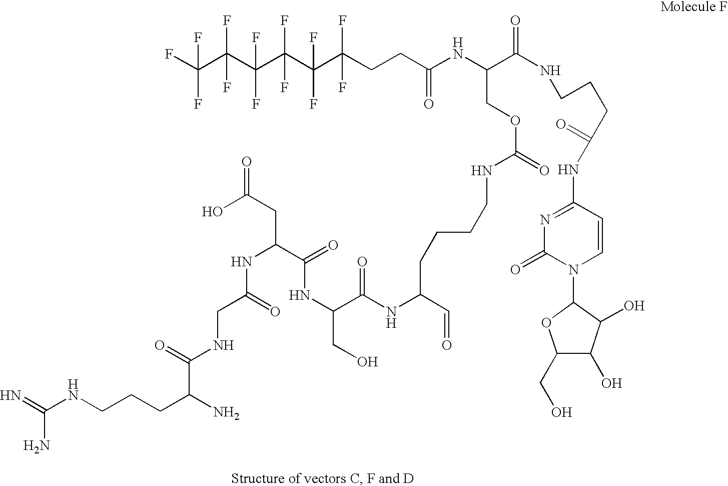

The antimitotic agents selected are merely models here, and simply illustrate the convenience of introducing and transporting a given antimitotic agent. This type of vector can and will also be used as an agent for the vectorization of substrates such as adriamycin (in this specific case, the molecule (Ic) contains a peptide fragment, either at X or at Pep, of Gly-Phe-Leu-Gly type), of 5-Fu (5-fluorouracil), of Melphalan, or of tyrosine kinase inhibitors such as imatinib mesylate (STI571, Glivec®), for example, or more generally of any anticancer agent that can be grafted onto these carriers. The presence of the hydrophobic fluorocarbon chain promotes the transmembrane passage. The release of the active principle is provided by hydrolysis of the peptide linkages by means of the appropriate cytoplasmic enzymes.

The first in vitro results already obtained fully validate this concept.

A subject of the invention is also the use of the compounds corresponding to formula (I) as defined above, for preparing a medicinal product.

In fact, it has been demonstrated that the compounds corresponding to formula (I) according to the present invention have a bioavailability and an ability to reach their biological targets that are greater than or equal to those of the compounds of the prior art.

This property makes it possible to envision the use of the molecules of the invention in varied fields:

in the therapeutic field, the products of the invention can be used for the prevention and/or treatment of all kinds of pathologies, in particular the various forms of cancer, and pathologies associated with oxidative stress and with the formation of oxygenated free-radical species.

Consequently, a subject of the invention is the pharmaceutical compositions comprising a compound according to the invention in a pharmaceutically acceptable carrier.

A subject of the invention is also the use of a compound of formula A, C, D or F, for preparing a pharmaceutical composition intended to prevent and/or treat cancer.

A subject of the invention is also the use of a compound of formula B, for preparing a pharmaceutical composition intended to detect the presence of cancerous cells.

A subject of the invention is also the use of a compound of formula E, for preparing a pharmaceutical composition intended to prevent and/or treat pathologies associated with oxidative stress and with the formation of oxygenated free-radical species, in particular immune and inflammatory diseases, ischemia-reperfusion syndrome, arteriosclerosis, Alzheimer's disease, Parkinson's disease, lesions due to UV and ionizing radiation, certain cancers such as melanomas, and cell aging.

The products of the invention can be administered by any route known to those skilled in the art, in particular by intravenous or intramuscular injection, or by oral or cutaneous administration. They can be used alone or in combination with other active agents. The dose thereof and the amount administered daily are adapted according to the activity measured for the molecule concerned and according to the weight of the patient;

in the cosmetics field, the compound of formula E can be used to prevent and/or treat the effects of aging.

A subject of the invention is therefore also a cosmetic composition comprising a compound of formula E in a cosmetically acceptable carrier.

Said composition may be intended for application to the skin or to the integuments (nails, hair).

It may be in the form of an aqueous or oily solution, of a water-in-oil or oil-in-water emulsion, of a triple emulsion or of an ointment.

The compounds of the invention can be introduced into any cosmetic composition for which a free-radical scavenger activity is desired: a skincare cream, an antisun product, a makeup-removing product, a mask for the skin or the hair, a shampoo, a makeup product such as a lipstick, a blusher, a foundation, a nail varnish, etc.

Due to their solubility in varied media, the compounds of the invention are easy to use and can be employed under very diverse conditions.

### EXPERIMENT SECTION

### 1/ Example 1

A—Preparation of Molecule A

2 g (4 mmol) of 1H, 1H, 2H, 2H-perfluorodecane azide compound dissolved in 30 ml of anhydrous methanol are subjected to hydrogenation in the presence of palladium-on-charcoal. After reaction for 4 hours, the medium is filtered through celite and the solvent is evaporated off under reduced pressure. The corresponding amine 2 is isolated without purification (quantitative yield).

The compound 2 is reacted again, in 30 ml of dichloromethane, in the presence of 2.2 g (4 mmol) of Boc-Lys-(Z)-OPhF₅ 3. The pH of the solution is brought to 8 by adding a few drops of DIEA.

After stirring for 16 hours at ambient temperature, the reaction medium is concentrated under reduced pressure.

The medium is purified by silica gel column chromatography (eluant: 3/7 ethyl acetate/cyclohexane). By means of crystallization from an ethyl acetate/hexane mixture, the fluorinated compound 4 (2.68 g; 3.24 mmol; 80%) is obtained in the form of a white powder.

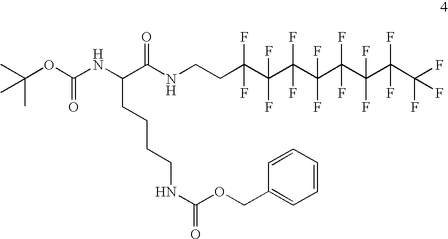

rf: 0.36 in 5/5 cyclohexane/ethyl acetate.

[α]_{D}=−8.2 (c: 1; CHCl₃).

Melting point: 86.5-88.3° C.

¹H NMR (250 MHz, CDCl₃): δ 7.36 (5H, s, CH arom), 6.85 (1H, m, NH amide), 5.27 (1H, d, J=6.65 Hz, NH urethane), 5.11 (2H, s, CH₂O), 4.99 (1H, t, J=5.8 Hz, NH urethane), 4.06 (1H, m, CHCO), 3.58 (2H, dd, J=6.5 Hz, CH₂NH), 3.20 (2H, dd, J=6.2 Hz, CH₂NH), 2.35 (2H, m, CH₂CF₂), 2.0 to 1.0 (15H, m, CH₃ from Boc and CH₂ Lys).

¹³C NMR (62.86 MHz, CDCl₃): δ 172.5 (CONH), 156.7; 155.9 (OCONH), 136.6 (C^{IV} arom.), 128.5; 128.1 (CH arom.), 80.4 (C^{IV}), 66.7 (CH₂OCONH), 54.4 (CHCO), 40.2 (CH₂NH), 31.9 (CH₂NH), 31.3 (CH₂), 30.7 (CH₂Rf), 29.5 (CH₂), 28.2 (CH₃ from tert-butyl), 22.4 (CH₂).

¹⁹F NMR (235 MHz, CDCl₃): δ −80.7 (3F, s, CF₃), −113.9 (2F, s, CF₂CH₂), −121.9 (6F, s, (CF₂)₃), −122.7 (2F, s, CF₂), −123.5 (2F, s, CF₂), −126.6 (2F, s, CF₂CF₃).

0.5 g (0.6 mmol) of compound 4 dissolved in 30 ml of dioxane is subjected to hydrogenation in the presence of palladium-on-charcoal.

After reaction for 15 hours, the medium is filtered through celite and the solvent is evaporated off under reduced pressure. The amine 5 obtained is reacted in dichloromethane in the presence of 0.21 g (0.44 mmol) of freshly prepared lactobionolactone and DIEA is added to the medium in order to bring the pH of the solution to 8.

After complete disappearance of the amine 5 (TLC), the reaction medium is concentrated under reduced pressure. 40 ml of a 1:1 acetic anhydride/pyridine mixture are added, under cold conditions, to the reaction crude. The stirring is maintained at ambient temperature for 18 hours and the reaction mixture is then poured onto 150 ml of 1N HCl. The aqueous phase is extracted 3 times with 50 ml of dichloromethane. The organic phase is respectively washed twice with 60 ml of 1N HCl and then with 60 ml of brine and, finally, dried over Na₂SO₄. The solvents are eliminated under reduced pressure and the crude is purified by flash chromatography on silica gel (eluant: 6/4 then 7/3 ethyl acetate/cyclohexane) so as to produce the compound 6 (0.55 g; 0.39 mmol; 65%) in the form of a white powder.

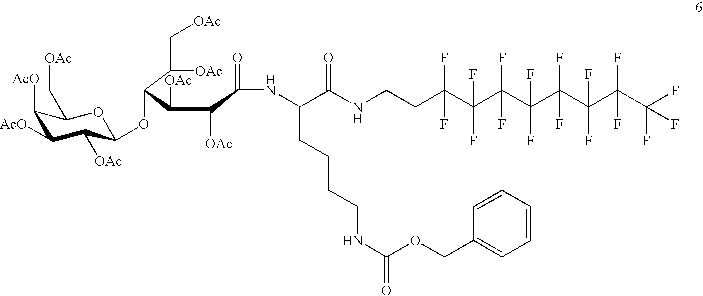

rf: 0.22 in 6/4 ethyl acetate/cyclohexane

[α]_{D}=+2.9 (c, 1; CHCl₃).

Melting point: 65° C. (beginning of decomposition).

¹H NMR (250 MHz, DMSO-d6): δ 8.07 (2H, m, NH), 7.34 (5H, s, CH arom.), 7.01 (1H, m, NH), 5.47 (1H, m, H from the sugar), 5.30 to 5.10 (2H, m, H from the sugar), 5.02 to 4.79 (5H, m, CH₂—O and H from the sugar), 4.50 to 3.90 (8H, m, H from the sugar and CHα from lysine), 3.38 (2H, m, CH₂—NH), 2.97 (2H, m, CH₂—NH), 2.30 (2H, m, CH₂—CF₂), 2.14, 2.09, 2.04, 2.01, 1.96, 1.92 (24H, 6s, CH₃ from acetyls), 1.65 to 1.10 (6H, m, CH₂ from lysine)

¹³C NMR (62.86 MHz, CDCl₃): δ 171.3 (CO—NH), 170.5, 170.5, 170.1, 170.0, 170.0, 169.7, 169.2 (7s, CO—O), 167.9 (CO—NH), 156.7 (O—CO—NH), 136.7 (C^{IV} arom.), 128.4, 128.0, 127.9 (CH arom.), 101.6 (CH-1′), 77.9 (CH-4), 72.7 (CH-2), 71.1, 70.9 (CH-5′ and CH-3′), 70.0 (CH-5), 69.4 (CH-3), 69.0 (CH-2′), 66.9 (CH-4′), 66.5 (CH₂—O), 61.6, 61.0 (CH₂-6 and CH₂-6′), 52.6 (CH—CO), 40.4 (CH₂—NH), 31.9 (CH₂—NH), 31.2 (CH₂—), 30.5 (CH₂—Rf), 29.1 (CH₂—), 22.2 (CH₂—), 20.6, 20.5, 20.4, 20.3 (CH₃ from acetyls)

¹⁹F NMR (235 MHz, DMSO-d6) δ −80.2 (3F, s, CF₃), −113.0 (2F, s, CF₂—CH₂), −121.4 (6F, s, 3CF₂), −122.2 (2F, s, CF₂), −123.0 (2F, s, CF₂), −125.4 (2F, s, CF₂—CH₂).

The benzyloxycarbonyl group of compound 6 is deprotected according to the experimental protocol already described when passing from compound 4 to compound 5. Using 0.5 g (0.36 mmol) of compound 6, the amine 7 is obtained with a quantitative yield.

The amine 7 obtained is reacted in 30 ml of dichloromethane in the presence of 0.21 g (0.44 mmol) of Z-Tyr-OPhF₅ (compound 8) and DIEA is added in order to bring the pH of the solution to 8.

After complete disappearance of the amine 7 (TLC), the reaction medium is concentrated under reduced pressure and purified by silica gel column chromatography (eluant: 7 ethyl acetate/3 cyclohexane). The compound 9 (0.32 g; 0.21 mmol; 58%) is obtained in the form of a white powder. 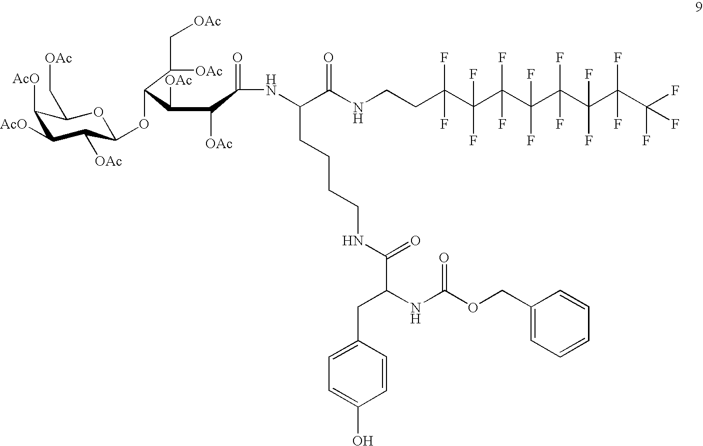
rf: 0.35 in 7 ethyl acetate/3 cyclohexane.

[α]_{D}=+1.5 (c: 1; CHCl₃).

Melting point: 37.6° C. (beginning of decomposition).

¹H NMR (250 MHz, DMSO-d6): δ 8.01 (2H, m, NH), 7.71 (1H, m, NH), 7.21 (5H, s, CH arom.), 7.05 (3H, m, NH, 2H arom tyr), 6.89 (2H, d, J=8.29 Hz 2H arom tyr), 5.22 (1H, d, J=5.35 Hz H from the sugar), 5.02 (2H, s, CH₂Ph), 4.99 (2H, s, CH₂Ph), 4.92 (2H, m, H from the sugar), 4.70 to 4.55 (5H, m, H from the sugar), 4.01 to 3.75 (9H, m, CHα tyr, CHα lys, CH₂NH, 5H from the sugar), 2.76 (2H, m, CH₂—NH), 2.16 (2H, m, CH₂—CF₂), 1.90 to 1.68 (24H, 6s, CH₃ from acetyls), 1.29 to 0.91 (6H, m, CH₂ from lysine)

¹³C NMR (62.86 MHz, CDCl₃): δ 171.85; 171.48 (CO—NH), 170.67, 170.32, 170.18, 170.0, 169.63; 169.42 (6s, CO—O), 156.29 (CO—NH), 137.47; 136.61; 135.54 (C^{IV} arom.), 130.71; 129.03; 128.80; 128.71; 128.12; 127.96; 121.17 (CH arom.), 101.07 (CH-1′), 78.69 (CH-4), 72.22 (CH-2), 70.86; 70.21 (CH-5′ and CH-3′), 70.06 (CH-5), 69.61 (CH-3), 69.3 (CH-2′), 67.60 (CH-4′), 65.69 (CH₂—O), 61.72, 61.48 (CH₂-6 and CH₂-6′), 56.62 (CHαtyr); 52.68 (CHαlys); 37.48 (CH₂—NH), 31.86 (CH₂—NH), 31.43 (CH₂—), 30.08 (CH₂—Rf), 29.09 (CH₂—), 22.77 (CH₂—), 21.08, 21.03, 20.94, 20.86, 20.76 (CH₃ from acetyls)

¹⁹F NMR (235 MHz, DMSO-d6): δ −80.19 (3F, s, CF₃), −113.38 (2F, s, CF₂—CH₂), −121.67 (6F, s, 3CF₂), −122.45 (2F, s, CF₂), −123.24 (2F, s, CF₂), −125.70 (2F, S, CF₂—CH₂).

Once again, according to the experimental protocol already described, 0.3 g (0.19 mmol) of compound 9 dissolved in 30 ml of ethanol is subjected to hydrogenation in the presence of palladium-on-charcoal.

The amine 10 obtained is reacted in dichloromethane in the presence of 0.107 g (0.23 mmol) of active ester of thalidomide 11 and DIEA is added in order to bring the pH of the solution to 8.

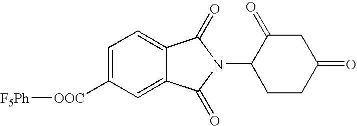

Rf=0.65 in 7 EtOAc/3 cyclohex.

[α_{D}]=+3.9 (c: 1; DMF).

¹H NMR (250 MHz, CDCl₃): 8.63 (1H, s, Ph); 8.61 (1H, d, Ph); 8.09 (1H, d, Ph); 5.04 (1H, m, NCH); 2.86 (3H, m, CH₂CO, CHCH₂CO); 2.19 (2H, m, CHCH₂).

¹⁹F NMR (235 MHz, CDCl₃): −152.6 (2F, d, CF); −156.67 (1F, t, CF); −161.87 (2F, t, CF).

¹³C NMR (62.86, DMSO): 177.97; 174.93; 171.64; 171.61; 170.96 (5 CO); 142.01; 140.94; 139.68; 136.89; 129.09; 128.64 (aromatic Cs); 54.44 (NCH); 36.13 (CH₂CO); 27.12 (NCHCH₂) 11

After complete disappearance of the amine 10 (TLC), the reaction medium is concentrated under reduced pressure and purified by silica gel column chromatography (eluant: 8/2 to 9/1 ethyl acetate/cyclohexane).

The compound 12 is obtained with a very low yield (11 mg; 6.4 μmol; 4.5%).

rf: 0.63 in ethyl acetate.

The deacetylation of the saccharide portion of the molecule is carried out at ambient temperature in methanol containing a catalytic amount of sodium methoxide.

After treatment on H⁺ resin (amberlite IRC 50), filtration and evaporation of the solvent, the deacetylated product A is isolated with a quantitative yield. 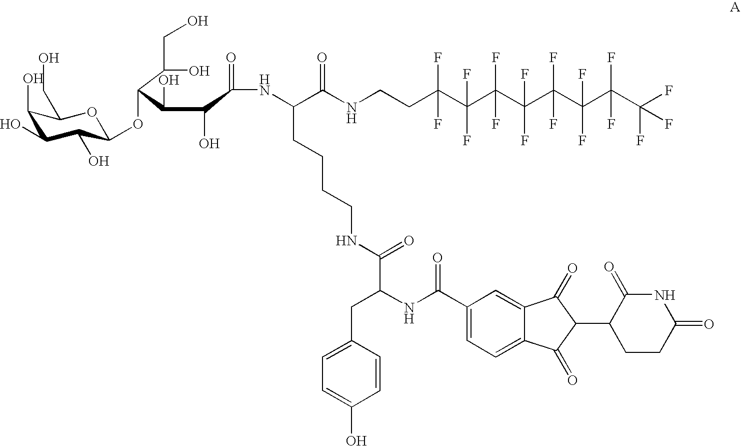
B—Biological Assays

Such a substrate exhibits no totally unacceptable toxicity on cell cultures of fibroblasts and of B16 melanomas. In vivo, the molecule is concentrated in the stroma of the tumor, which makes it possible to visualize it very clearly (cf. Table 1).

TABLE 1
Radioactivity measured in various organs of mice carrying B16
melanomas after IP injection of molecule A (10 μCi/animal).

| Tissue | 15 min | 1 h |
|---|---|---|
| Tumor (stroma) | 4.5 ± 0.7 | 5.4 ± 0.2 |
| Tumor (center) | 2.6 ± 0.5 | 2.7 ± 0.5 |
| Blood | 5.9 ± 1.2 | 3.4 ± 0.7 |
| Liver | 4.5 ± 1.1 | 2.2 ± 0.8 |
| Kidney | 4.6 ± 0.5 | 4.0 ± 0.9 |
| Thyroid | 5.6 ± 0.8 | 9.8 ± 1.7 |

The ongoing research will make it possible to specify its effectiveness, compared with thalidomide alone, in blocking tumor development. The first results obtained in vascular growth assays on chick embryos (chick aortic ring assays) showed the effectiveness of this structure in inhibiting microvessel growth. Molecule A is found to be effective at 20 μM and completely blocks the development at 200 μM, whereas, at such concentrations, thalidomide is found to be ineffective.

This first result demonstrates the general innocuity of the proposed structure and its possible advantage for diagnosis, thalidomide here being merely an example of an active principle.

### 2/Example 2

### Preparation of Molecule E

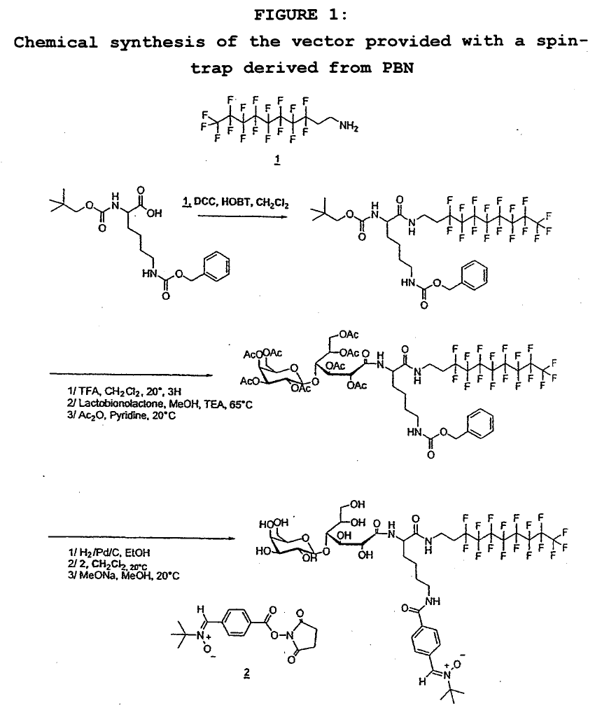

This synthesis example is illustrated in FIG. 1.
Synthesis of the Active Ester HOOCPBN

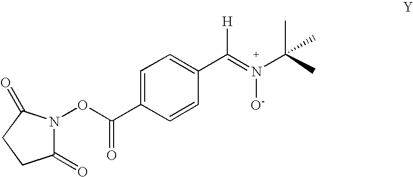

N-(tert-Butyl)hydroxylamine acetate is dissolved in a saturated aqueous sodium carbonate solution. The hydroxylamine is extracted with ether. The organic phase is dried over Na₂SO₄ and the solvent is then eliminated under reduced pressure so as to produce free N-tert-butylhydroxylamine in the form of pulverulent white crystals.

1.00 g of 4-carboxybenzaldehyde (6.67 mmol—1 equiv.) and the tip of a spatula of 4 Å molecular sieve are suspended, under an argon atmosphere, in 5 ml of degassed anhydrous ethanol. 0.570 g of hydroxylamine (6.37 mmol—0.95 equiv.) in solution in 5 ml of ethanol are added to the benzaldehyde solution, and the medium is brought to 60° C., in the dark. After 18 hours, 0.200 g of hydroxylamine (2.25 mmol—0.33 equiv.) are added to the medium and stirring is continued for a further 18 hours. The reaction mixture is filtered through a layer of celite, the solvent is eliminated under reduced pressure and the crude is purified by flash chromatography on silica gel (eluant: 6:4 ethyl acetate/cyclohexane). After recrystallization from a methanol/ether mixture, the nitrone X (0.590 g-2.67 mmol —40%) is obtained in the form of a white powder.

Molar mass (C₁₂H₁₅NO₃): 221.3 g·mol⁻¹

Melting point: 214.5-215.7° C.

¹H NMR (250 MHz, DMSO-d6): δ 8.42 (2H, m, J=8.5 Hz, H arom.), 7.95 (3H, m, H arom. and CH═N(O)), 1.51 (9H, s, CH₃ from tert-butyl).

¹³C NMR (62.86 MHz, DMSO-d6): δ 166.9 (CO), 135.3 (C^{IV} arom), 131.0 (CH═N(O)), 129.2 (CH arom.), 128.3 (C^{IV} arom), 127.9 (CH arom.), 71.1 (C^{IV}), 27.8 (CH₃ from tert-butyl).

UV (MeOH, nm): λₘₐₓ=287.

0.260 g of nitrone X (1.18 mmol—1 equiv.) is dissolved, under an argon atmosphere, in 15 ml of dioxane. 0.290 g of DCC (1.41 mmol—1.2 equiv.) and 0.16 g of HOSu (1.41 mmol—1.2 equiv.) are added to the reaction medium. After stirring for 48 hours, the reaction medium is filtered through sintered glass (porosity 4) and the solvent is then eliminated under reduced pressure. After purification by flash chromatography on silica gel (eluant: 6:4 ethyl acetate/cyclohexane) and then recrystallization from an ethyl acetate/hexane mixture, the compound Y (0.25 g-0.79 mol—67%) is obtained in the form of a white powder.

Molar mass (C₁₆H₁₈N₂O₅): 318.3 g·mol⁻¹

Melting point: 177.4-178.3° C.

¹H NMR (250 MHz, CDCl₃): δ 8.37 (2H, m, J=8.6 Hz, H arom.), 8.12 (2H, d, J=8.6 Hz, H arom.), 7.65 (1H, s, CH═N(O)), 2.89 (4H, s, CH₂—CO), 1.61 (9H, s, CH₃ from tert-butyl).

¹³C NMR (62.86 MHz, CDCl₃): δ 169.3, 161.3 (CO), 136.8 (C^{IV} arom), 130.7 (CH arom.), 128.6 (C^{IV} arom), 128.6 (CH arom.), 125.5 (CH═N(O)), 72.2 (C^{IV}), 28.4 (CH₂—CO), 25.7 (CH₃ from tert-butyl).
Synthesis of [5-tert-butoxycarbonylamino-5-(3,3,4,4,5,-5,6,6,7,7,8,8,9,9,10,10,10-heptadecafluorodecyl-carbamoyl)pentyl]carbamic acid benzyl ester 3

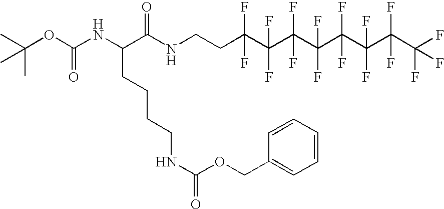

2.26 g of azide C₈F₁₇CH₂CH₂N₃ (4.62 mmol—1 equiv.) are dissolved in 20 ml of ether. The medium is brought to 0° C. and 300 mg of palladium-on-charcoal (10%—65 mg/mmol) are added fractionwise. After stirring for 6 hours in the hydrogenation bomb (pressure 8 bar), the medium is filtered through a layer of celite and the solvents are eliminated under reduced pressure. The amine C₈F₁₇CH₂CH₂NH₂ 2 is obtained without purification.

1.79 g of Boc-Lys (Z)OH (4.70 mmol—1 equiv.), 1.07 g of DCC (5.18 mmol—1.1 equiv.) and 0.64 g of HOBt (4.70 mmol—1 equiv.) are dissolved in 20 ml of anhydrous dichloromethane. After stirring for 15 minutes, the amine 2 in solution in 10 ml of anhydrous dichloromethane is added to the mixture. The stirring is continued for 24 hours. The reaction crude is filtered through sintered glass (porosity 4) and the solvents were then eliminated under reduced pressure. After purification by flash chromatography on silica gel (eluant: 7:3 to 6:4 cyclohexane/ethyl acetate), the fluorinated compound 3 (3.11 g-3.70 mmol—79%) is obtained in the form of a white powder.

Molar mass (C₂₉H₃₂F₁₇N₃O₅): 825.6 g·mol⁻¹

Melting point: 86.5-88.3° C.

¹H NMR (250 MHz, CDCl₃): δ 7.36 (5H, s, CH arom.), 6.85 (1H, m, NH amide), 5.27 (1H, d, J=6.65 Hz, NH urethane), 5.11 (2H, s, CH₂—O), 4.99 (1H, t, J=5.8 Hz, NH urethane), 4.06 (1H, m, CH—CO), 3.58 (2H, dd, J=6.5 Hz, CH₂—NH), 3.20 (2H, dd, J=6.2 Hz, CH₂—NH), 2.35 (2H, m, CH₂—CF₂), 2.0 to 1.0 (15H, m, CH₃ from Boc and CH₂ lys).

¹³C NMR (62.86 MHz, CDCl₃): δ 172.5 (CO—NH), 156.7, 155.9 (O—CO—NH), 136.6 (C^{IV} arom.), 128.5, 128.1 (CH arom.), 80.4 (C^{IV}), 66.7 (CH₂—O—CO—NH), 54.4 (CH—CO), 40.2 (CH₂—NH), 31.9 (triplet, CH₂—NH), 31.3 (CH₂), 30.7 (CH₂—Rf), 29.5 (CH₂), 28.2 (CH₃ from tert-butyl), 22.4 (CH₂).

¹⁹F NMR (235 MHz, DMSO-d6): δ −80.7 (CF₃, s), −113.9 (CF₂—CF₃, s), −121.9 (3 CF₂, m), −122.7 (CF₂, s), −123.5 (CF₂, s), 126.0 (CF₂—CH₂, s).

[α]_{D}=−8.2 (c, l, CHCl₃).
Synthesis of LactoLys (Z)C₈F₁₇(OAc)₈ 5

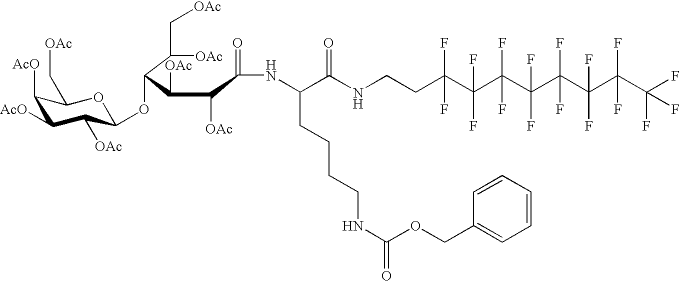

2.03 g of compound 3 (2.45 mmol—1 equiv.) are dissolved in 20 ml of anhydrous dichloromethane. The medium is brought to 0° C. and 40 ml of an 8.5:1.5 CH₂Cl₂/TFA mixture are added dropwise while keeping the temperature at ° C. throughout the addition. After stirring for 4 hours, the solvents are eliminated under reduced pressure. The crude is taken up in ether and then evaporated in order to eliminate the residual traces of TFA by co-evaporation. The operation is repeated several times and produces the free amine 4.

In parallel, 1.15 g of lactobionic acid (3.19 mmol—1.3 equiv.) are suspended in 40 ml of a 1:1 methoxyethanol/toluene mixture acidified with 3 drops of TFA.

After evaporation at 45° C. under reduced pressure, the medium is taken up in 30 ml of a 2:1 methoxyethanol/toluene mixture and then evaporated to dryness. The latter operation is repeated twice so as to produce lactobionolactone.

The lactobionolactone and the amine 4 are dissolved, under an argon atmosphere, in 40 ml of methanol. The pH of the solution is brought to 9 by adding TEA, and the medium is then brought to reflux for 24 hours. After elimination of the methanol under reduced pressure, 40 ml of a 1:1 acetic anhydride/pyridine mixture are added, under cold conditions, to the crude. The stirring is maintained for 18 hours and the reaction mixture is then poured onto 150 ml of 1N HCl. The aqueous phase is extracted with 3 times 50 ml of dichloromethane. The organic phase is respectively washed with twice 60 ml of 1N HCl and then with 60 ml of brine and, finally, dried over Na₂SO₄. The solvents are eliminated under reduced pressure and the crude is purified by flash chromatography on silica gel (eluant: 6:4 to 7:3 ethyl acetate/cyclohexane) so as to produce the compound 5 (2.23 g-1.59 mmol—65%) in the form of a white powder.

Molar mass (C₅₂H₆₀F₁₇N₃O₂₂): 1402.0 g·mol⁻¹

Melting point: 65° C. (beginning of decomposition).

¹H NMR (250 MHz, DMSO-d6): δ 8.07 (2H, m, NH), 7.34 (5H, s, CH arom.), 7.01 (1H, m, NH), 5.47 (1H, m, H from the sugar), 5.30 to 5.10 (2H, m, H from the sugar), 5.02 to 4.79 (5H, m, CH₂—O and H from the sugar), 4.50 to 3.90 (8H, m, H from the sugar and CH from lysine), 3.38 (2H, m, CH₂—NH), 2.97 (2H, m, CH₂—NH), 2.30 (2H, m, CH₂—CF₂), 2.14, 2.09, 2.04, 2.01, 1.96, 1.92 (24H, 6s, CH₃ from acetyls), 1.65 to 1.10 (6H, m, CH₂ from lysine).

¹³C NMR (62.86 MHz, CDCl₃): δ 171.3 (CO—NH), 170.5, 170.5, 170.1, 170.0, 170.0, 169.7, 169.2 (7s, CO—O), 167.9 (CO—NH), 156.7 (O—CO—NH), 136.7 (C^{IV} arom.), 128.4, 128.0, 127.9, (CH arom.), 101.6 (CH-1′), 77.9 (CH-4), 72.7 (CH-2), 71.1, 70.9 (CH-5′ and CH-3′), 70.0 (CH-5), 69.4 (CH-3), 69.0 (CH-2′), 66.9 (CH-4′), 66.5 (CH₂—O), 61.6, 61.0 (CH₂-6 and CH₂-6′), 52.6 (CH—CO), 40.4 (CH₂—NH), 31.9 (triplet, CH₂—NH), 31.2 (CH₂—), 30.5 (triplet, CH₂—Rf), 29.1 (CH₂—), 22.2 (CH₂—), 20.6, 20.5, 20.4, 20.3 (5s, CH₃ from acetyls).

¹⁹F NMR (235 MHz, DMSO-d6): δ −80.2 (CF₃, s), −113.0 (CF₂—CF₃, s), −121.4 (3 CF₂, s), −122.2 (CF₂, s), −123.0 (CF₂, s), −125.4 (CF₂—CH₂, s).

[α]_{D}=+2.9 (c, 1, CHCl₃).
Synthesis of LactoLys(PBN)C₈F₁₇(OAc)₈ 7

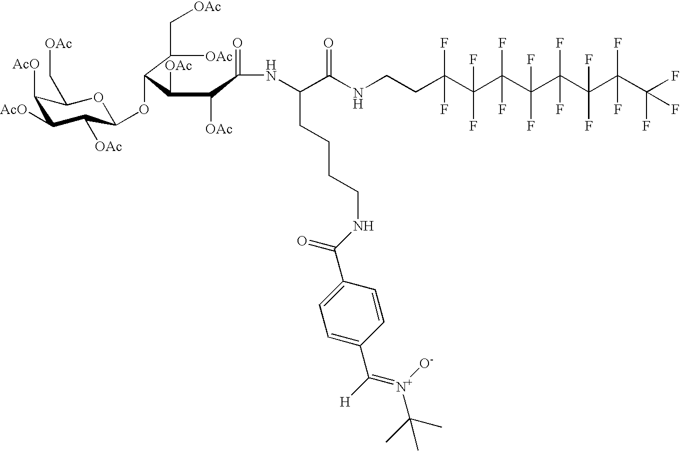

0.400 g of compound 5 (0.28 mmol —1 equiv.) is dissolved in 10 ml of dioxane. The medium is brought to 0° C. and 0.190 g of palladium-on-charcoal (10%—65 mg/mmol) are added fractionwise. After stirring for 20 hours in the hydrogenation bomb (pressure 8 bar), the medium is filtered through a layer of celite and the solvents are eliminated under reduced pressure. The amine 6 is obtained in the form of a white powder without purification.

The amine 6 is dissolved, under a stream of argon, in 5 ml of anhydrous dichloromethane. 0.090 g of active ester X (0.28 mmol—1 equiv.) is added to the medium and the pH is brought to 9 by adding DIEA.

The stirring is continued under an argon atmosphere for 24 hours. The solvents are evaporated off under reduced pressure and the crude is purified by flash chromatography on silica gel (eluant: ethyl acetate). A further purification by size exclusion chromatography on Sephadex LH-20 resin (eluant: 1:1 dichloromethane/ethanol) makes it possible to obtain the nitrone 7 (0.230 g-0.156 mmol—54%) in the form of a white powder.

Molar mass (C₅₆H₆₇F₁₇N₄O₂₂): 1471.1 g·mol⁻¹

Melting point: 75° C. (beginning of decomposition).

¹H NMR (250 MHz, CDCl₃): δ 8.36 (2H, d, J=8.3 Hz, H arom.), 7.92 (2H, d, J=8.4 Hz, H arom.), 7.68 (1H, s, CH═N(O)), 6.94 (3H, m, NH), 5.51 (1H, dd, J=2 Hz and J=4.4 Hz, H-4′), 5.32 (2H, m, H-2 and H-3), 5.17 to 4.95 (3H, m, H-2′, H-5 and H-3′), 4.62 (1H, d, J=7.7 Hz, H-1′), 4.50 (1H, dd, J=2.7 Hz and J=12.5 Hz, H from the sugar), 4.33 (1H, m, CH from the lysine), 4.18 (1H, dd, J=1.6 Hz and J=6.3 Hz, H from the sugar), 4.12 to 3.87 (4H, m, H from the sugar and H-5′), 3.50 (2H, m, CH₂—NH), 3.39 (2H, m, CH₂—NH), 2.35 (2H, m, CH₂—CF₂), 2.15, 2.09, 2.08, 2.04, 2.02, 2.00, 1.96 (24H, 7s, CH₃ from acetyls), 1.60 (11H, m, CH₃ from tert-butyl and CH₂ Lys), 1.85 (2H, m, CH₂ Lys), 1.35 (2H, m, CH₂ Lys).

¹³C NMR (62.86 MHz, CDCl₃): δ 171.5 (CO—NH), 170.6, 170.5, 170.3, 170.3, 170.0, 169.7, 169.32 (7s, CO—O), 168.2 (CO—NH), 167.1 (CO—NH), 135.2 (C^{IV} arom.), 133.7 (C^{IV} arom.), 129.3 (CH═N(O)), 128.6, 127.3 (CH arom.), 101.7 (CH-1′), 78.5 (CH-4), 72.9 (CH-2), 71.4 (C^{IV}), 71.0, 70.9 (CH-5′ and CH-3′), 68.9 (CH-5), 69.3 (CH-3), 69.0 (CH-2′), 66.9 (CH-4′), 61.6, 61.1 (CH₂-6 and CH₂-6′), 52.7 (CH—CO), 39.4 (CH₂—NH), 31.9 (m, CH₂—NH), 31.1 (CH₂—), 30.5 (triplet, CH₂—Rf), 28.7 (CH₂—), 28.2 (CH₃ from tert-butyl), 22.2 (CH₂—), 20.7, 20.7, 20.7, 20.6, 20.5, 20.5, 20.4, (7s, CH₃ from acetyls).

¹⁹F NMR (235 MHz, CDCl₃): δ −80.7 (s, CF₃), −114.2 (s, CF₂—CF₃), −121.9 (s, 3 CF₂), −122.7 (s, CF₂), −123.5 (s, CF₂), −126.1 (s, CF₂—CH₂).

[α]_{D}=+1.6 (c, 1, CHCl₃).

### 3/ Example 3

### A—Synthesis of Compound B

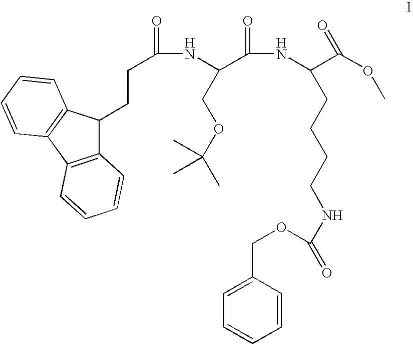

0.99 g (3×10⁻³ mol) of Cl⁻⁺H₃N Lys(Z) OMe are dissolved in 10 ml of dichloromethane, in a 100 ml round-bottomed flask. The pH is brought to 8 with TEA. 1.15 g (3×10⁻³ mol, 1 eq.) of Fmoc Ser(OtBu) OH are then added, along with 1.72 g (3.9×10⁻³ mol, 1.3 eq.) of BOP.

The pH is maintained at 8 throughout the reaction. The medium is kept stirring at ambient temperature for 24 hours. Once the reaction is complete (TLC), the organic phase is washed with 1N HCl and then with NaHCO₃ in order to reestablish a pH of 7. The organic phase is dried over Na₂SO₄, filtered, and then evaporated under reduced pressure. Crystallization can be carried out from a dichloromethane/Et₂O mixture. 1.93 g of the compound 1 are obtained in the form of a white powder.

Yield: 98%.

Rf=0.58 in 6 ethyl acetate/4 cyclohexane.

Degradation temperature: 80.6° C. [α]^{D}₂₀=+17.9 (c, 1, CH₂Cl₂).

¹H NMR in CDCl₃:

δ=7.75 (2H, d, ar Fmoc); 7.60 (2H, d, ar Fmoc); 7.43-7.21 (5H, m, ar Fmoc, NH Lys); 7.31 (5H, s, ar Z Lys); 5.78 (1H, d, NH Ser); 5.08 (2H, s, CH₂, Z Lys); 4.84 (1H, t, NH Z Lys); 4.60 (1H, m, CH Lys); 4.39 (2H, d, CH₂ Fmoc); 4.24 (2H, m, CH Ser, CH Fmoc); 3.81 (1H, dd, CH₂ Ser); 3.73 (3H, s, OCH₃ Lys); 3.39 (1H, dd, CH₂ Ser); 3.17 (2H, CH₂—NH Lys); 1.85-1.29 (6H, m, CH—CH₂—CH₂—CH₂ Lys); 1.22 (9H, s, tBu Ser).

¹³C NMR in CDCl₃:

δ=172.94 (CO Lys); 170.88 (CO Ser); 157.08; 156.67 (2 O—CO—NH Fmoc and Z); 144.52; 144.36; 141.93; 137.23; 129.13; 128.71; 128.35; 127.71; 125.76; 120.62 (C ar Fmoc and Z); 74.96 (C(CH₃)₃ Ser); 67.80; 67.24 (CH₂ Fmoc and CH₂ Z); 62.39 (C*—CH₂ Ser); 54.93 (C*—CH₂ Ser); 52.99; 52.74 (OCH₃ Lys and C*—CH₂ Lys); 47.76 (CH Fmoc); 41.27 (CH₂—NH—CO—O Lys); 32.74 (CH₂—CH₂—NH—CO—O Lys); 30.02 (C*—CH₂ Lys); 27.99 (C(CH₃)₃ Ser); 22.92 (C*—CH₂—CH₂ Lys).

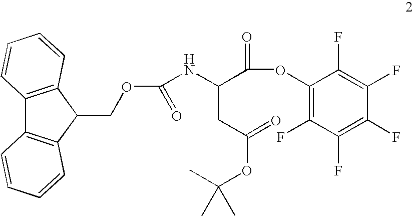

0.68 g (1.53×10⁻³ mol) of Fmoc Asp(OtBu)OH is dissolved, in 10 ml of dichloromethane, along with 0.34 g (1.83×10⁻³ mol, 1.2 eq) of pentafluorophenol and 0.38 g (1.83×10⁻³ mol, 1.2 eq) of DCC, in a 50 ml round-bottomed flask. The reaction is left stirring at ambient temperature for 15 hours. After filtration, the medium is concentrated under reduced pressure. The residual oil is chromatographed on silica gel (eluant: 2/8: ethyl acetate/cyclohexane). Crystallization is carried out from ethyl acetate/hexane. 760 mg of 2 are obtained in the form of a white powder.

Yield: 86.2%.

Melting point: 94.6-95.8° C.

[α]^{D}₂₀=−2.3 (c, 1, CH₂Cl₂).

¹H NMR in CDCl₃:

δ=7.77 (2H, d, ar Fmoc); 7.61 (2H, d, ar Fmoc); 7.43-7.28 (4H, m, ar Fmoc); 5.98 (1H, d, NH); 4.98 (1H, m, CH Asp); 4.49-4.23 (3H, m, CH₂ Fmoc, CH Fmoc); 3.15 (1H, dd, CH₂ Asp); 2.90 (1H, dd, CH₂ Asp); 1.48 (9H, s, tBu Asp).

¹³C NMR in CDCl₃:

δ=170.35; 168.09 (2 CO—O); 156.55 (O—CO—NH Fmoc); 144.43; 144.26; 142.01; 128.47; 127.78; 125.79; 120.71 (C ar Fmoc); 143.82-136.34 (CF); 83.39 (C(CH₃)₃ Asp); 68.21 (CH₂ Fmoc); 50.97 (C*—CH₂ Asp); 47.76 (CH Fmoc); 38.31 (C*—CH₂ Asp); 28.68 (C(CH₃)₃ Asp).

¹⁹F NMR in CDCl₃:

δ=−152.22 (2F); −157.36 (1F); −162.10 (2F)

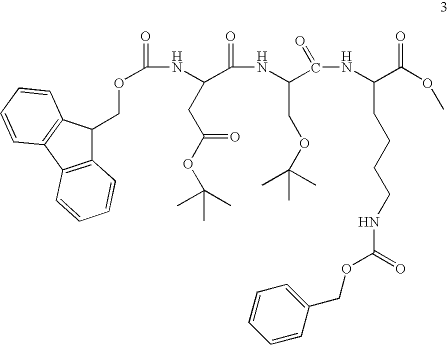

Before the coupling between the dipeptide and the activated amino acid, a step consisting of deprotection of the compound 2 is necessary. For this, 1.3 g (1.97 mmol) of the dipeptide 1 are dissolved in 15 ml of a 10% v/v piperidine/dichloromethane mixture. The medium is left to stir for two hours at ambient temperature and is then washed with a separating funnel, with a 1N HCl solution. The organic phase is then washed with a saturated sodium hydrogen carbonate solution. The organic phases are subsequently dried over sodium sulfate and then concentrated under reduced pressure. The coupling can then be carried out.

The dipeptide deprotected above is dissolved in 20 ml of dichloromethane, in a 100 ml single-necked round-bottomed flask, in the presence of 1.138 g (1.97 mmol, 1 eq) of compound 2 added to the round-bottomed flask. The reaction takes place at ambient temperature under a stream of nitrogen, in the dark, at a pH of 8 fixed with DIEA. After 15 hours, the reaction is complete (TLC). The reaction medium is concentrated and the residue is chromatographed on silica gel in a 5/5 ethyl acetate/cyclohexane eluant mixture. After evaporation of the solvent, 970 mg of compound 3 are obtained in the form of a translucent gel.

Yield: 59.2%.

¹H NMR in CDCl₃: δ=7.78 (2H, d, ar Fmoc); 7.61 (2H, d, ar Fmoc); 7.46-7.23 (6H, m, ar Fmoc, NH Lys, NH Ser); 7.36 (5H, s, H ar Z Lys); 5.93 (1H, d, NH Asp); 5.11 (2H, s, CH₂ Z Lys); 4.91 (1H, t, NH Z Lys); 4.57 (2H, m, CH Asp, CH Lys); 4.48-4.42 (3H, m, CH Ser, CH₂ Fmoc); 4.26 (1H, t, CH Fmoc); 3.85 (1H, dd, CH₂ Ser); 3.74 (3H, s, OCH₃ Lys); 3.41 (1H, dd, CH₂ Ser); 3.17 (2H, CH₂—NH Lys); 2.88 (1H, dd, CH₂ Asp); 2.72 (1H, dd, CH₂ Asp); 1.48 (9H, s, tBu Asp); 1.84-1.28 (6H, m, CH—CH₂—CH₂—CH₂ Lys); 1.22 (9H, s, tBu Ser).

¹³C NMR in CDCl₃: δ=172.96; 171.74; 171.14; 170.52 (CO Lys, CO Ser, 2 CO Asp); 157.06; 156.65 (2 O—CO—NH Fmoc and Z); 144.32; 141.94; 137.28; 129.14; 128.71; 128.41; 127.76; 125.72; 120.65 (C ar Fmoc and Z); 82.59 (C(CH₃)₃ Asp); 74.80 (C(CH₃)₃ Ser); 68.04; 67.88 (CH₂ Fmoc and CH₂ Z); 61.61 (C*—CH₂ Ser); 53.99 (C*—CH₂ Ser); 52.95; 52.70 (OCH₃ Lys and C*—CH₂ Lys); 52.04 (C*—CH₂ Asp); 47.74 (CH Fmoc); 41.32 (CH₂—NH—CO—O Lys); 38.25 (C*—CH₂ Asp); 32.55 (CH₂—CH₂—NH—CO—O Lys); 29.93 (C*—CH₂ Lys); 28.67 (C(CH₃)₃ Asp); 27.97 (C(CH₃)₃ Ser); 22.99 (C*—CH₂—CH₂ Lys).

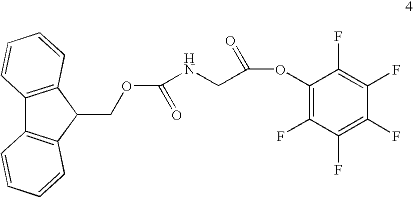

1 g (3.36×10⁻³ mol) of Fmoc Gly OH, 0.681 g of pentafluorophenol (3.7×10⁻³ mol, 1.1 eq) and 0.764 g of DCC (3.7×10⁻³ mol, 1.1 eq) are dissolved in 10 ml of dichloromethane. The medium is left at ambient temperature for 24 hours. The reaction medium is subsequently filtered, and the filtrate is then concentrated by evaporation under reduced pressure. The residue is subjected to flash chromatography on silica gel with a 5/5 ethyl acetate/cyclohexane eluant mixture. Crystallization is carried out from an ethyl acetate/cyclohexane mixture. 955 mg of 4 are obtained in the form of a white powder. Yield: 61.3%.

Melting point: 156.3-157.4.

[α]^{D}₂₀=−1.3 (c, 1, CH₂Cl₂).

¹H NMR in CDCl₃: δ=7.82 (2H, d, ar Fmoc); 7.65 (2H, d, ar Fmoc); 7.47-7.25 (4H, m, ar Fmoc); 5.40 (1H, t, NH); 4.54-4.42 (5H, m, CH₂ Fmoc, CH Fmoc, CH₂ Gly).

¹³C NMR in CDCl₃: δ=167.14 (CO—O Gly); 156.84 (NH—CO—O); 144.31; 142.00; 128.46; 127.77; 125.66; 120.70 (C ar Fmoc); 68.14; (CH₂ Fmoc); 47.73 (CH Fmoc); 42.87 (CH₂ Gly).

¹⁹F NMR in CDCl₃: δ=−152.44 (2F); −157.25 (1F); −161.96 (2F).

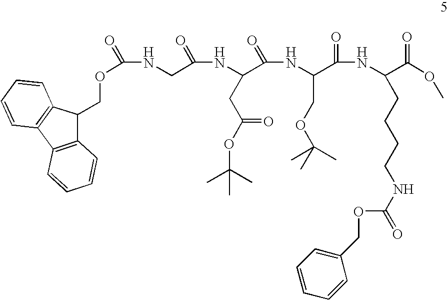

This synthesis is preceded by deprotection of the tripeptide according to the same protocol as for the synthesis of this tripeptide (compound 3). 500 mg (6×10⁻⁴ mol) of 3 are deprotected. Once deprotected, this peptide is dissolved in 10 ml of dichloromethane in a 100 ml round-bottomed flask. 278 mg (6×10⁻⁴ mol, 1 eq) of compound 4 are added to the reaction medium. The reaction is carried out at ambient temperature under a stream of nitrogen, and is maintained at pH 8 with DIEA. After 16 hours, the reaction is complete (TLC). After evaporation of the medium under reduced pressure, the residual oil is chromatographed on silica gel with a 6/3 ethyl acetate/cyclohexane eluant mixture. From an ethyl acetate/cyclohexane mixture, 426 mg of 5 are obtained in the from of a white powder. Yield: 80%.

Degradation temperature: 63.7° C.

[α]^{D}₂₀=+4.6 (c, 1, CH₂Cl₂).

¹H NMR in CDCl₃: δ=7.76 (2H, d, ar Fmoc); 7.60 (2H, d, ar Fmoc); 7.45-7.22 (7H, m, ar Fmoc, NH Lys, NH Ser, NH Asp,); 7.34 (5H, s, ar Z Lys); 5.83 (1H, d, NH Gly); 5.14 (1H, t, NH Z Lys) 5.07 (2H, s, CH₂ Z Lys); 4.82 (1H, m, CH Asp); 4.57 (1H, m, CH Lys); 4.45-4.35 (3H, m, CH Ser, CH₂ Fmoc); 4.20 (1H, t, CH Fmoc); 3.89 (2H, m, CH₂ Gly); 3.75 (1H, dd, CH₂ Ser); 3.68 (3H, s, OCH₃ Lys); 3.39 (1H, dd, CH₂ Ser); 3.15 (2H, CH2—NH Lys); 2.88 (1H, dd, CH₂ Asp); 2.68 (1H, dd, CH₂ Asp); 1.41 (9H, s, tBu Asp); 1.91-1.29 (4H, m, CH—CH₂—CH₂ Lys); 1.22 (9H, s, tBu Ser).

¹³C NMR in CDCl₃: δ=173.00; 171.90; 170.75; 170.49; 169.76 (CO Lys, CO Ser, 2 CO Asp, CO Gly); 157.31; 157.13 (2 O—CO—NH Fmoc and Z); 144.45; 141.98; 137.27; 129.19; 128.78; 128.42; 127.78; 125.77; 120.68 (C ar Fmoc and Z); 82.78 (C(CH₃)₃ Asp); 74.78 (C(CH₃)₃ Ser); 68.05; 67.30 (CH₂ Fmoc and CH₂ Z); 61.60 (C*—CH₂ Ser); 54.19 (C*—CH₂ Ser); 53.01; 52.72 (OCH₃ Lys and C*—CH₂ Lys); 50.28 (C*—CH₂ Asp); 47.76 (CH Fmoc); 45.26 (CH₂ Gly); 41.38 (CH₂—NH—CO—O Lys); 37.68 (C*—CH₂ Asp); 32.58 (CH₂—CH₂—NH—CO—O Lys); 29.99 (C*—CH₂ Lys); 28.67 (C(CH₃)₃ Asp); 28.00 (C(CH₃)₃ Ser); 23.03 (C*—CH₂—CH₂— Lys).

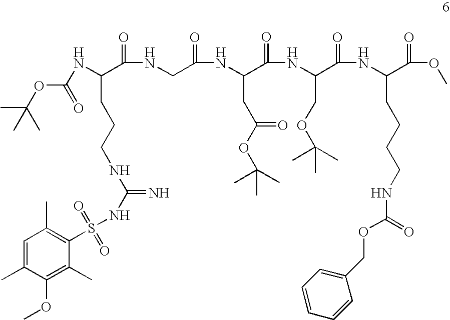

The deprotection of the pentapeptide 5 is carried out under the same conditions as in the synthesis of compound 1. 400 mg (4.5×10⁻⁴ mol) of the tetrapeptide are deprotected. Once the deprotection is complete, this tetrapeptide is dissolved in 15 ml of dichloromethane in a 50 ml single-necked round-bottomed flask. 219 mg (4.5×10⁻⁴ mol, 1 eq) of Boc Arg(Mtr) OH and 188 mg (5.85×10⁻⁴ mol, 1.3 eq) of TBTU are added. The reaction is carried out at ambient temperature under a stream of nitrogen, in the dark, and is maintained at pH 8 with DIEA. After 16 hours, the reaction is complete (TLC). After evaporation under reduced pressure, the residual oil is chromatographed on silica gel using a 9/1 ethyl acetate/cyclohexane eluant. Crystallization is carried out from ethyl acetate/cyclohexane. 417 mg of 6 are obtained in the form of a white powder. Yield: 92.5%.

Degradation point: 80.2° C.

[α]^{D}₂₀=−2.9 (c, 1, CH₂Cl₂).

¹H NMR in CDCl₃: δ=7.64 (1H, d, NH Asp); 7.53 (1H, d, NH Gly); 7.49-7.29 (7H, m, ar Z Lys, NH Lys, NH Ser); 6.54 (1H, s, H ar Mtr); 6.34-6.15 (3H, m, 3 NH guanidine Arg); 5.65 (1H, d, NH Boc Arg); 5.24 (1H, t, NH Z Lys); 5.10 (2H, s, CH₂ Z Lys); 4.77 (1H, m, CH Asp); 4.46 (2H, m, CH Lys, CH Ser); 4.22 (1H, m, CH Arg); 3.89-3.70 (3H, m, CH₂ Gly, 1H from CH₂ Ser); 3.84 (3H, s, OCH₃ Mtr Arg); 3.71 (3H, s, OCH₃ Lys); 3.50 (1H, dd, CH₂ Ser); 3.21 (2H, m, CH₂—CH₂—NH Arg); 3.17 (2H, CH₂—NH Lys); 2.90-2.64 (8H, m, 2 CH₃ Mtr Arg, CH₂ Asp); 2.15 (3H, s, CH₃ Mtr Arg); 1.84-1.19 (37H, m, tBu Asp, tBu Ser, tBu Boc Arg, CH—CH₂—CH₂Lys, CH—CH₂—CH₂ Arg).

¹³C NMR in CDCl₃: δ=174.32; 174.13; 172.85; 171.48; 171.35; 170.92; 170.18 (CO Lys, CO Ser, 2 CO Asp, CO Gly, CO Arg, NH—C—NH Arg); 158.96; 157.20; 156.55 (2 O—CO—NH Boc and Z, C—OCH₃ Mtr Arg); 139.07; 137.25; 137.06; 134.19; 129.07; 128.87; 128.61; 125.30, 112.28 (C ar Mtr and Z); 82.37 (C(CH₃)₃ Asp); 80.53 (C(CH₃)₃ Arg); 74.63 (C(CH₃)₃ Ser); 67.09 (CH₂ Z); 60.96 (C*—CH₂ Ser); 55.99 (C—OCH₃ Mtr Arg); 54.57; 54.23 (C*—CH₂ Ser, C*—CH₂ Arg); 52.87; 52.74 (OCH₃ Lys and C*—CH₂ Lys); 50.37 (C*—CH₂ Asp); 43.92 (CH₂ Gly); 41.25; 40.75 (CH₂—NH—CO—O Lys, CH₂—NH—C—NH Arg); 37.59 (C*—CH₂ Asp); 32.27 (CH₂—CH₂—NH—CO—O Lys); 30.33; 29.83 (C*—CH₂ Lys, C*—CH₂ Arg); 28.94; 28.56; 27.86 (C(CH₃)₃ Asp, C(CH₃)₃ Ser, C(CH₃)₃ Arg); 25.93 (C*—CH₂—CH₂ Arg); 24.74 (C*—CH₂—CH₂ Lys); 23.06; 18.95; 12.52 (3 CH₃ Mtr Arg).

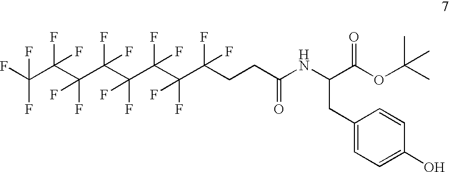

500 mg (1.01×10⁻³ mol) of C₈F₁₇CH₂CH₂COOH, 278 mg of H₂N TYR(OH) OtBu (1.01×10⁻³ mol, 1 eq) and 251 mg of DCC (1.2×10⁻³ mol, 1.2 eq) are dissolved in 10 ml of DMF, in a 50 ml round-bottomed flask. The reaction is carried out at ambient temperature for 24 hours under a stream of nitrogen, in the dark, and is maintained at pH 8 with DIEA. After evaporation of the DMF under reduced pressure, the residue is chromatographed on silica gel using a 2/8 ethyl acetate/cyclohexane eluant.

The product can be crystallized from ethyl acetate/n-heptane. 550 mg of 7 are obtained in the form of a white powder. Yield: 76%.

Melting point:

[α]^{D}₂₀=+30.0 (c, 1, CH₂Cl₂).

¹H NMR in CDCl₃: δ=6.98 (2H, d, 2H ar Tyr); 6.72 (2H, d, 2H ar Tyr); 6.04 (1H, m, NH Tyr); 4.72 (1H, m, CH Tyr); 3.02 (2H, m, CF₂—CH₂—CH₂—CO); 2.47 (4H, m, CF₂—CH₂—CH₂—CO, CH₂ Tyr); 1.44 (9H, s, tBu).

¹³C NMR in CDCl₃: δ=170.94 (CO—O); 169.52 (CO—NH); 155.13 (C—OH Tyr); 130.70 (C ar Tyr); 127.82 (C—CH ar Tyr); 115.49 (CH—C—OH ar Tyr); 122.5-106.3 (CF₃(CF₂)₇); 82.92 (C—(CH₃)₃)); 53.93 (NH—C*—CO); 37.32 (C*—CH₂); 27.16 (CH₂—CF₂); 25.02 (CH₂—CO).

¹⁹F NMR in CDCl₃: δ=−82.17; −115.50; −122.63; −123.49; −124.25; −127.06.

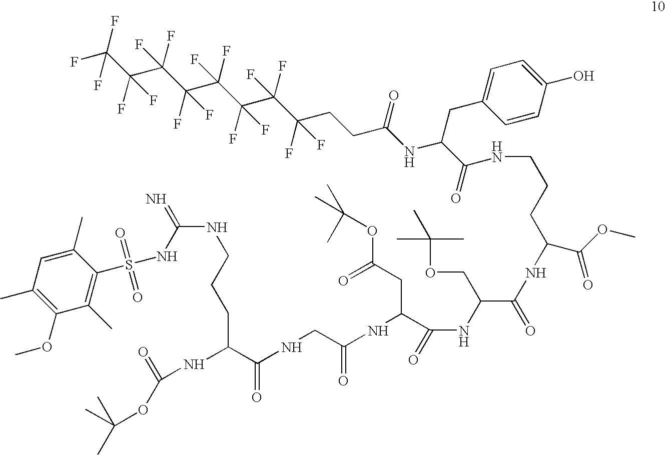

380 mg (5.34×10⁻⁴ mol) of compound 7 are dissolved, under cold conditions, in a TFA/CH₂Cl₂ (3/7) solution. After stirring for 2 hours, the deprotection is complete. The reaction medium is subsequently concentrated and then precipitated from ether several times. After evaporation, a white powder is obtained (compound 8).

150 mg (1.32×10⁻⁴ mol) of compound 6 are dissolved in methanol. 8 mg of palladium-on-charcoal at 10% are added under cold conditions. The reaction medium is placed under 8 atmospheres of hydrogen. After 2 h 30 min, the reaction is complete. The mixture is filtered through celite 521 and the filtrate is concentrated under reduced pressure (compound 9).

Compound 9 (1.32×10⁻⁴ mol) is dissolved in 10 ml of DMF, in a 50 ml round-bottomed flask. 95 mg of compound 8 (1.45×10⁻⁴ mol, 1.1 eq) and 76 mg of TBTU (1.72×10⁻⁴ mol, 1.3 eq) are added. The reaction is carried out at ambient temperature for 24 hours under a stream of nitrogen, in the dark, and is maintained at pH 8 with DIEA. The reaction medium is concentrated under reduced pressure. The residue is chromatographed on silica gel using an ethyl acetate eluant. 141 mg of product 10 are obtained in the form of a white powder. Yield 65.3%.

Degradation temperature: 76.8° C.

[α]^{D}₂₀=−5.4 (c, 1, CH₃OH).

¹H NMR in CD₃OD: δ=7.06 (2H, d, 2H ar Tyr); 6.74 (1H, s, H ar Mtr); 6.69 (2H, d, 2H ar Tyr); 4.80-4.53 (4H, m, CH Asp, CH Lys, CH Ser CH Tyr); 4.04 (1H, m, CH Arg); 3.88-3.85 (5H, m, CH₂ Gly, OCH₃ Mtr Arg); 3.72-3.63 (5H, m, OCH₃ Lys, CH₂ Ser); 3.22-2.31 (18H, m, CH₂—CH₂—NH Arg, CH₂—NH Lys, 2 CH₃ Mtr Arg, CH₂ Asp, CF₂—CH₂—CH₂—CO, CF₂—CH₂—CH₂—CO, CH₂ Tyr); 2.15 (3H, s, CH₃ Mtr Arg); 1.85-1.20 (37H, m, tBu Asp, tBu Ser, tBu Boc Arg, CH—CH₂—CH₂—CH₂ Lys, CH—CH₂—CH₂ Arg).

¹³C NMR in CDCl₃: δ=179.69; 174.13-173.95; 172.32; 172.07; 171.29; 171.10; 170.77; 170.21 (CO Lys, CO Ser, 2 CO Asp, CO Gly, CO Arg, NH—C—NH Arg, CO—NH Tyr, CO—NH, C₈F₁₇CH₂CH₂CONH); 158.49; 156.80; 155.94 (O—CO—NH Boc, C—OCH₃ Mtr Arg, C—OH ar Tyr); 138.10; 136.49; 133.45; 129.97; 127.54; 124.31; 114.81; 111.41 (C ar Mtr, C ar Tyr); 81.22; 79.47 (C(CH₃)₃ Asp, C(CH₃)₃ Arg); 73.42 (C(CH₃)₃ Ser); 61.12 (C*—CH₂ Ser); 54.61; 52.24; 51.31; 49.91 (C—OCH₃ Mtr Arg, C*—CH₂ Ser, C*—CH₂ Arg, NH—C*—CO Tyr, OCH₃ Lys and C*—CH₂ Lys, C*—CH₂ Asp); 42.29; 40.08; 38.60; 37.15; 36.69 (CH₂ Gly, CH₂—NH—CO—O Lys, CH₂—NH—C—NH Arg, C*—CH₂ Asp, C*—CH₂ Tyr); 30.77; 28.92; 28.25; 27.40; 26.96; 26.34; 25.95 (CH₂—CH₂—NH—CO—O Lys, C*—CH₂ Lys, C*—CH₂ Arg, C(CH₃)₃ Asp, C(CH₃)₃ Ser, C(CH₃)₃ Arg, CH₂—CF₂, C*—CH₂—CH₂ Lys); 22.94; 22.53 (1 CH₃ Mtr Arg, CF₂—CH2-CH2-CO); 17.42; 10.71 (2 CH₃ Mtr Arg).

Compound 10, subjected to the action of a solution of trifluoroacetic acid in CH₂Cl₂ in the presence of thioanizole (3; 5; 2) for 24 h, produces compound B, which is isolated in pure form after precipitation by adding ether to the solution, and chromatography on a Sephadex G50 column (eluant: H₂O). After lyophilization, the product B is in the form of a white powder.

B—Biological Assays

The cytotoxicity of this molecule was tested on B16 melanoma cells; no toxicity could be measured up to concentrations of greater than 100 μM. After labeling with iodine 125, this molecule, injected intravenously into a batch of mice carrying a melanoma, accumulates in the stroma of the tumor and then diffuses slowly within the tumor (cf Table 2).

TABLE 2
Radioactivity measured in mice carrying B16 melanoma
after IV injection of molecule B (10 μCi/animal).

| Tissue | 15 min | 30 min | 1 h |
|---|---|---|---|
| Tumor (stroma) | 3.9 ± 0.8 | 3.8 ± 0.9 | 4.2 ± 0.6 |
| Tumor (center) | 1.7 ± 0.6 | 1.8 ± 0.3 | 2.4 ± 0.4 |
| Blood | 5.8 ± 0.8 | 4.5 ± 0.8 | 3.6 ± 0.7 |
| Liver | 4.8 ± 0.8 | 2.2 ± 0.6 | 2.4 ± 0.7 |
| Kidney | 8.9 ± 1.6 | 5.9 ± 0.6 | 3.5 ± 0.6 |
| Thyroid | 5.9 ± 1.2 | 10.2 ± 2.5 | 15.3 ± 5.3 |

### 4/Example 4

Methyl N-(t-butoxycarbonyl)-N-^{γ}-(2,3,6-trimethyl-4-methoxybenzenesulfonyl)-L-arginylglycinyl-O-(t-butyl)-L-aspartyl-O-(t-butyl)-L-serinyl-N-^{ε}-((4,4,5,-5,6,6,7,7,8,8,9,9,10,10,11,11,11-heptadecafluoro-undecanoyl)-N-^{ε}-(4-(4-[bis(2-chloroethyl)amino]phenyl)-butyramido)-L-lysinyl-L-tyrosinamido)-L-lysinate

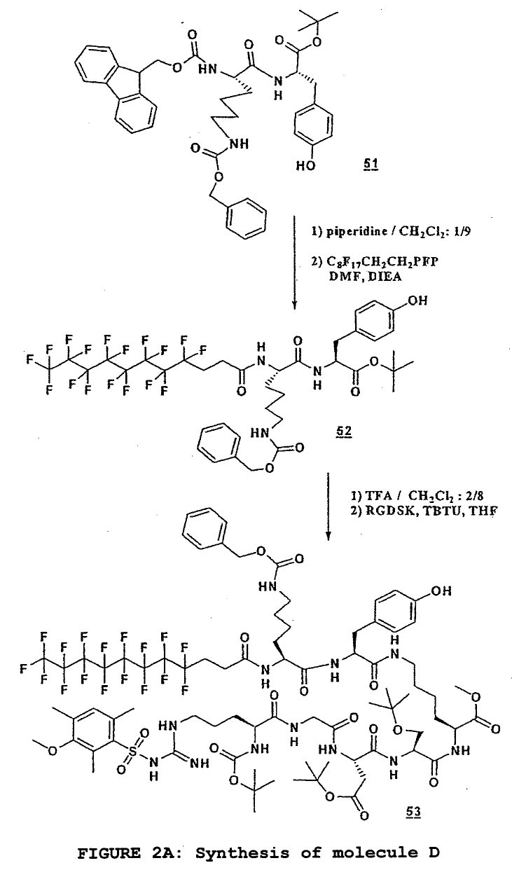

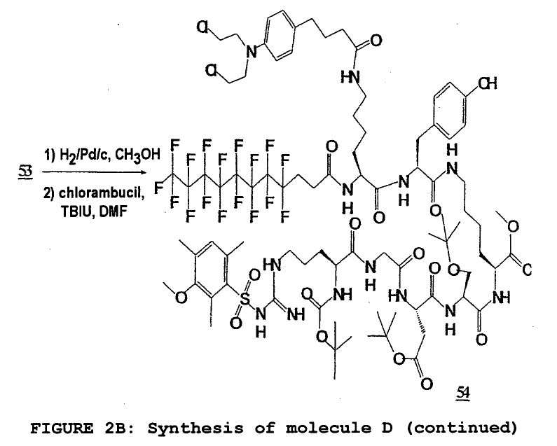

The synthesis of compound D is comparable to those described above for the above compounds and is summarized in FIGS. 2A and 2B.

Physicochemical Characteristics of D

Rf: 0.49 in ethyl acetate/methanol: 98/2.

MM: 2051.87 g·mol⁻¹.

Degradation temperature: 134° C.

[α]^{D}₂₀=−0.77 (c, 0.1, CH₃OH).

MS (FAB): m/z 2052 [M+H⁺], 2074 [M+Na]⁺.

¹H NMR (CD₃OD):

δ 7.03 (4H, m, 2H arom. Tyr, 2H arom. Chloramb.); 6.67 (5H, m, 2H arom. Tyr, 2H arom. Chloramb., H arom Mtr); 4.81; 4.47; 4.20; 4.01 (6H, 3m, H_{α} Tyr, 2H_{α} Lys, H_{α} Asp, H_{α} Ser, H_{α} Arg); 3.82 (3H, s, CH₃ ether Mtr Arg); 3.68 (3H, s, CH₃ methyl ester Lys); 3.83-3.60 (12H, m, 2H_{α} Gly, 2H_{β} Ser, 8H Chloramb.); 3.14-2.75 (10H, m, 4H_{ε} Lys, 2H_{δ} Arg, 2H_{β} Asp, 2H_{β} Tyr); 2.67-2.48 (8H, m, 2H_{α} and 2H_{β} fluorinated chain, 2H_{α} Chloramb., 2H_{δ} Chloramb.); 2.67; 2.61 (6H, 2m, 2CH₃ Mtr Arg); 2.13 (5H, m, 2H_{β} Chloramb., CH₃ Mtr Arg); 1.93-1.16 (16H, m, 4H_{β} Lys, 4H_{γ} Lys, 4H_{δ} Lys, 2H_{β} Arg, 2H_{γ} Arg); 1.44; 1.42; 1.16 (27H, 3s, 9 CH₃ tert-butyl ester Asp, tert-butyl ether Ser, tert-butyl urethane Arg).

¹³C NMR (CD₃OD):

δ 174.67; 174.14; 172.57; 172.31; 171.80; 171.75; 171.26; 170.80; 170.17 (CO Tyr, 2CO Lys, CO Ser, 2 CO Asp, CO Gly, CO Arg, CO fluorinated chain, CO Chloramb.); 158.46; 156.80; 156.67; 155.93 (CO urethane Boc, C—OCH₃ arom. Mtr Arg, C—OH arom. Tyr, C guanidine Arg); 144.55 (C—N arom. Chloramb.); 138.10; 136.48; 133.42; 130.29; 129.97; 129.22; 127.51; 124.29; 114.83; 112.06; 111.38; 110.73 (C arom. Mtr, C arom. Tyr, C arom. Chloramb.); 81.17; 79.43 (C tert-butyl ester Asp, C tert-butyl urethane Arg); 73.40 (C tert-butyl ether Ser); 61.14 (C_{β} Ser); 54.96; 54.60; 54.53; 53.89; 53.14; 52.26; 51.31; 49.90 (CH₃ methyl ether Mtr Arg, C_{α} Ser, C_{α} Arg, C_{α} Tyr, CH₃ methyl ester Lys, 2C_{α} Lys, C_{α} Asp, 2C—N Chloramb.); 42.30; 40.29; 38.58; 36.67; 36.52; 35.20 (C_{α} Gly, 2C_{ε} Lys, C_{δ} Arg, C_{β} Asp, C_{β}Tyr, 2C—Cl Chloramb.); 33.85; 31.02; 30.69; 28.91; 28.67; 28.15; 27.69; 27.41; 26.96; 26.36; 25.82; 25.46 (2C_{δ} Lys, 2C_{β} Lys, C_{β} Arg, C_{γ} Arg, 9 CH₃ tert butyl Asp, Ser, Arg, C_{α}—CF₂, 2C_{γ} Lys, C_{α}, C_{γ} Chloramb.); 23.00; 22.66 (C_{β} fluorinated chain, C_{γ} Chloramb.); 22.52; 17.47; 10.74 (3 CH₃ methyl Mtr Arg).

¹⁹F NMR (CD₃OD):

δ −82.26 (3F); −115.39 (2F); −122.77 (6F); −123.62 (2F); −124.30 (2F); −127.17 (2F)

### 5/Example 5

Methyl L-arginyl-glycinyl-L-aspartyl-L-serinyl-N-^{ε}-(—O—(N-⁴-(N-(4,4,5,5,6,6,7,7,8,8,9,9-tridecafluoro-nonanoyl)-L-serinyl)-1-β-D-arabinofuranosylcytosine)-oxycarbonyl)-L-lysinate 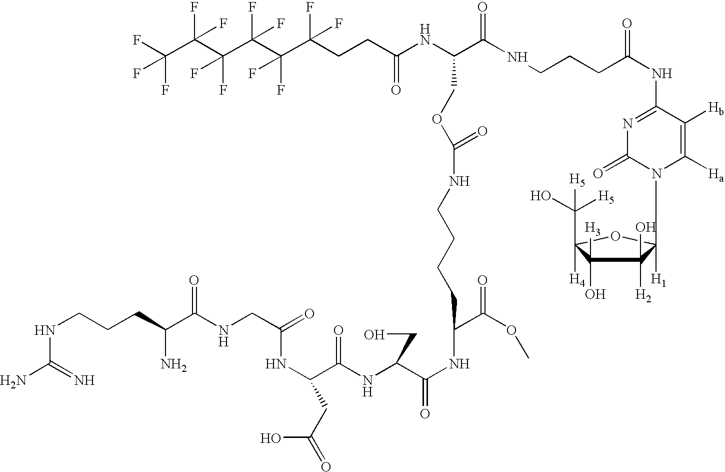
Procedure:

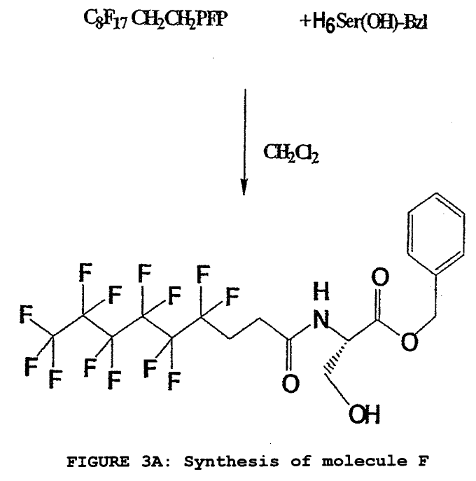

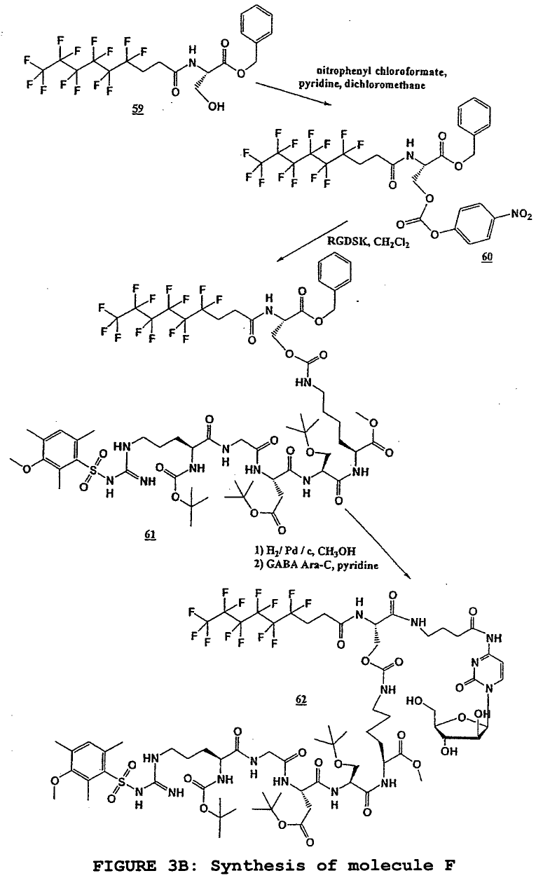

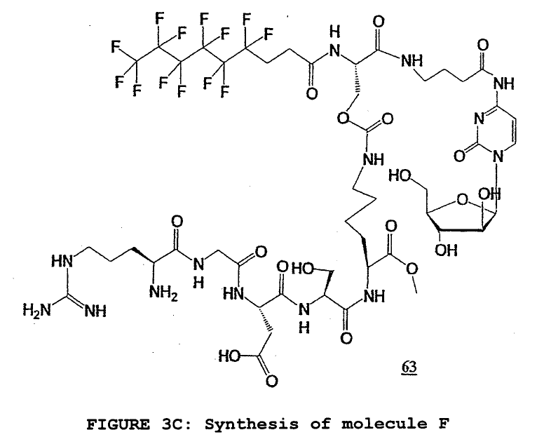

The process for preparing F is identical to the previous processes; the synthesis is summarized in FIGS. 3A, 3B and 3C.

Physicochemical Characteristics of F

MM: 1390.45 g·mol⁻¹. MS (FAB): m/z 1391 [M+H]⁺.

¹H NMR (DMSO, D₆):

δ 8.66 (1H, s, H acid Asp); 8.27-8.16; 7.94-7.84; 7.65; 7.21-6.92 (15H, 3m, Hₐ, NH GABA, 2NH Ser, 4H guanidine Arg, NH₂ Arg, NH Gly, NH Asp, 2NH Lys, NH Ara-C); 5.84 (1H, m, H_{b}); 5.31 (2H, m, 1H_{α} Ser, H₁); 4.92 (1H, m, H_{α} Asp); 4.37-3.71 (17H, m, H_{α} Lys, H_{α} Ser, H_{α} Arg, 2H_{α} Gly, 4H_{β} Ser, H₂, H₃, H₄, CH₃ methyl ester Lys, 2H₅); 3.18-2.53 (14H, m, 2H_{ε} Lys, CH₂—NH GABA, 4OH, 2H_{β} Asp, 2H_{α} and 2H_{β} fluorinated chain); 2.23 (2H, m, CH₂—CO GABA); 1.46-1.03 (14H, m, 2H_{β}, 2H_{γ}, 2H_{δ} Lys, 2H_{β}, 2H_{γ}, 2H_{δ} Arg, CH₂ GABA).

¹³C NMR (DMSO, D₆):

δ 173.9; 173.6; 173.4; 172.8; 171.1; 170.4; 170.1; 169.6; 169.1; 168.6 (CO Lys, 2 CO Ser, 2 CO Asp, CO Gly, CO Arg, CO fluorinated chain, CO GABA); 162.6; 157.4; 156.2; 155.0 (C guanidine Arg, CO urethane Ser, N═C—NH, N—CO—N); 147.2 (Cₐ); 94.8 (C_{b}); 87.5; 86.2 (C₁, C₄); 76.6; 75.0 (C₂, C₃); 65.4; 62.0; 61.5 (2 C_{β} Ser, C₅); 55.6; 53.0; 52.5; 52.3; 51.7; 50.1 (2 C_{α} Ser, C_{α} Arg, CH₃ methyl ester Lys, C_{α} Lys, C_{α} Asp); 42.5 (C_{α} Gly); 40.8; 38.5; 37.3; 34.8; 34.1; 30.9; 29.4; 29.0; 26.3; 24.8; 24.3; 23.0 (C_{ε} Lys, C_{δ} Arg, CH₂—NH GABA, C_{β} Asp, C_{δ} Lys, C_{β} Lys, CH₂—CO GABA, C_{β} Arg, C_{α} and C_{β} fluorinated chain, C_{γ} Arg, CH₂ GABA, C_{γ} Lys).

¹⁹F NMR (DMSO, D₆):

δ −82.30 (3F); −115.51 (2F); −122.81 (6F); −123.82 (2F); −124.46 (2F); −127.21 (2F).

1. A compound corresponding to formula (I) below: 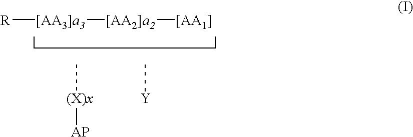
in which:
AP represents an active principle capable of acting on a biological target;
x represents an integer chosen from 0 and 1;
X represents a peptide chain comprising from 1 to 5 amino acids;
AA₁, AA₂ and AA₃, which may be identical or different, each represent an amino acid;
a₂ and a₃, which may be identical or different, each represent an integer chosen from 0 and 1;
R represents a group chosen from:
any molecule capable of being recognized by the target of the active principle AP, and
a hydrophilic agent for modulating the HLB balance of the molecule of formula (I), R being chosen from monosaccharides, aminated derivatives of sugars, polysaccharides, natural or synthetic hormones, peptides, antibodies, polyethers and polyols,
Y represents a fluorinated C₄-C₁₂ hydrocarbon-based chain containing a group 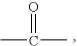
—NH—, —O—CO—NH—, S or O that allows its attachment either to one of the ends of the peptide chain [AA₃]ₐ₃-[AA₂]ₐ₂-[AA₁], or to the side chain of one of the amino acids AA₁, AA₂ or AA₃;
the linkage between AP-(X)ₓ and the chain [AA₃]ₐ₃-[AA₂]ₐ₂-[AA₁] occurring via the side chain of one of the amino acids AA₁, AA₂ or AA₃ or at the end of the peptide chain.

2. The compound as claimed in claim 1, wherein the active principle is chosen from those that have anticancer activity, or free-radical scavenger, anti-inflammatory, antiseptic, analgesic, neuroleptic or antifungal activity.

3. The compound as claimed in claim 1, wherein the active principle is a linear, branched or cyclic molecule containing from 1 to 30 carbon atoms, one or more unsaturations, one or more aromatic rings, and one or more functions chosen from: —O—, —S—, —OH, —SH, —Cl, —F, —Br,

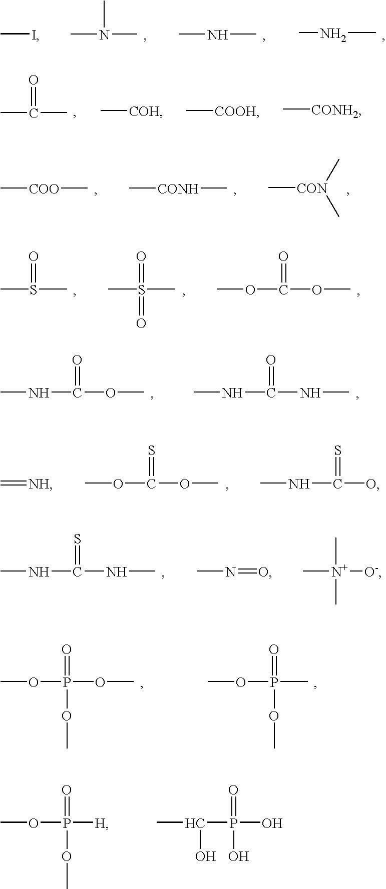

4. The compound as claimed in claim 1, wherein the amino acid attached to AP-(X)ₓ- or to Y via its side chain is chosen from those containing an acid, amide, amine, thiol or alcohol function on their side chain.

5. The compound as claimed in claim 1, wherein the spacer arm X comprises 1 to 3 amino acids.

6. The compound as claimed in claim 1, wherein R is a peptide chosen from antibody fragments or epitopes having a pronounced affinity for the AP's biological target.

7. The compound as claimed in claim 6, which contains at least one peptide sequence chosen from the Arg-Gly-Asp sequence.

8. The compound as claimed in claim 1, wherein R consists of a poly(ethylene oxide) chain comprising from 5 to 30 ethylene oxide units or of a polyol consisting of an alkyl chain comprising from 4 to 16 carbon atoms and from 4 to 16 hydroxyl groups.

9. The compound as claimed in claim 1, wherein R is chosen from: glucose, fructose, mannose, galactose, ribose, glucosamine, lactose, cellobiose, maltose, lactobionamide and sucrose.

10. The compound as claimed in claim 1, wherein at least one of the spacer arms X, of the peptide chain [AA₃]ₐ₃-[AA₂]ₐ₂-[AA₁] and of R contains at least one tyrosine residue.

11. The compound as claimed in claim 1, wherein the fluorinated hydrocarbon-based chain Y is chosen from those corresponding to the formula A-Y′ in which A represents a group chosen from: 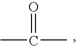
—NH—, —O—CO—NH—, S and O and Y′ represents a molecule corresponding to the formula —(CH₂)ₜ—(CF₂)ᵣF, in which r and t represent two integers with: 12≧r+t≧4.

12. A biologically active molecule comprising a fragment of formula (II): 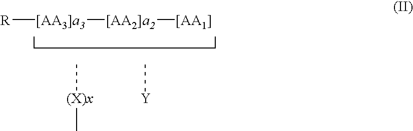
in which x represents an integer chosen from 0 and 1;
X represents a peptide chain comprising from 1 to 5 amino acids;
AA₁, AA₂ and AA₃, which may be identical or different, each represent an amino acid;
a₂ and a₃, which may be identical or different, each represent an integer chosen from 0 and 1;
R is chosen from monosaccharides, aminated derivatives of sugars, polysaccharides, polyethers, polyols, peptides, natural or synthetic hormones, and antibodies;
Y represents a fluorinated C₄-C₁₂ hydrocarbon-based chain containing a group 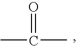
—NH, —O—CO—NH—, S or O that allows its attachment either to one of the ends of the peptide chain [AA₃]ₐ₃-[AA₂]ₐ₂-[AA₁], or to the side chain of one of the amino acids AA₁, AA₂ or AA₃, and at least one of the spacer arms X, of the peptide chain [AA₃]ₐ₃-[AA₂]ₐ₂-[AA₁] and of R contains at least one tyrosine residue.

13. The compound as claimed in claim 1, corresponding to formula (Ia): 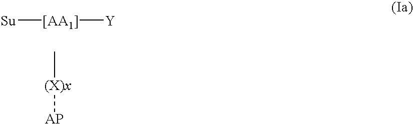
in which:
Su represents a group chosen from a monosaccharide, an aminated monosaccharide derivative, a polysaccharide, a polyol or a polyether;
AA₁ represents an amino acid carrying an acid, amine, alcohol or thiol function on its side chain, by means of which it is attached either to (X)ₓ-AP or to Y; AA₁ is attached to Su and either to (X)ₓ-AP, or to Y, via its N- and C-terminal ends;
AP represents an active principle capable of acting on a biological target;
x represents an integer chosen from 0 and 1;
X represents a peptide chain comprising from 1 to 5 amino acids;
Y represents a fluorinated C₄-C₁₂ hydrocarbon-based chain containing a function chosen from 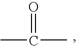
—NH, —O—CO—NH—, S and O that allows its attachment either to one of the ends of the amino acid AA₁, or to the side chain of AA₁.

14. The compound as claimed in claim 13, wherein one or more of the conditions below are verified:
Su represents a monosaccharide or a polysaccharide;
X represents a spacer arm that is peptide in nature, containing at least one tyrosine residue;
AA₁ represents an amino acid chosen from arginine and lysine;
Y represents a fluorinated C₆-C₁₂ hydrocarbon-based chain containing from 5 to 23 fluorine atoms, attached to the amino acid AA₁ via an —NH— function.

15. The compound as claimed in claim 14, wherein the active principle is chosen from molecules capable of blocking the angiogenic process, in particular thalidomide.

16. The compound as claimed in claim 15, corresponding to formula A:

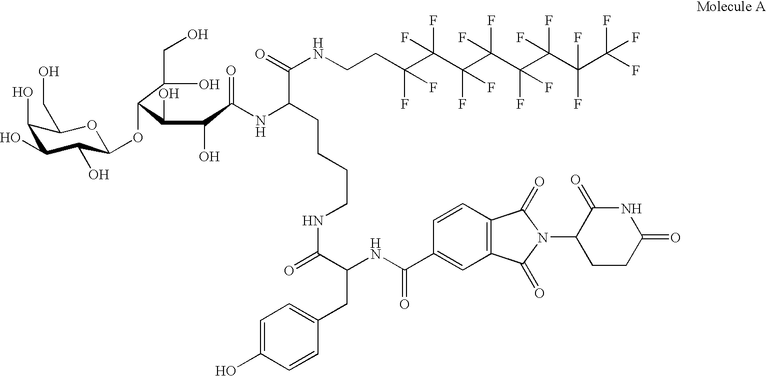

17. The compound as claimed in claim 15, wherein the active principle AP is chosen from free-radical scavengers, in particular N-benzylidene-tert-butylamine oxide derivatives.

18. The compound as claimed in claim 17, corresponding to formula E:

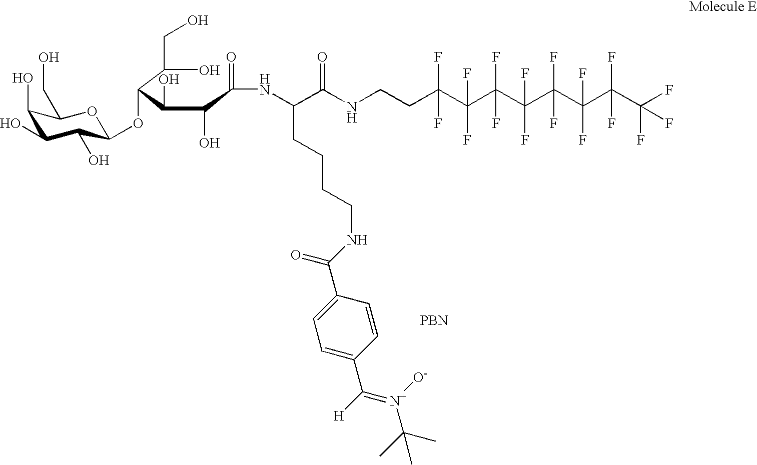

19. The compound as claimed in claim 12, corresponding to formula (Ib):
Pep-[AA₁]-Y (Ib)
in which:
AA₁ represents an amino acid carrying an acid, amine, alcohol or thiol function on its side chain,
Y represents a fluorinated C₄-C₁₂ hydrocarbon-based chain containing a function chosen from 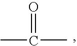
—NH, —O—CO—NH—, S and O that allows its attachment either to one of the ends of the amino acid AA₁, or to the side chain of AA₁,
Pep represents a peptide chain containing from 2 to 10, preferably from 4 to 6, amino acids, at least one of Pep and of AA₁ containing at least one tyrosine unit.

20. The compound as claimed in claim 19, wherein Pep contains an arginine-glycine-aspartic acid sequence.

21. The compound as claimed in claim 1, corresponding to formula B:

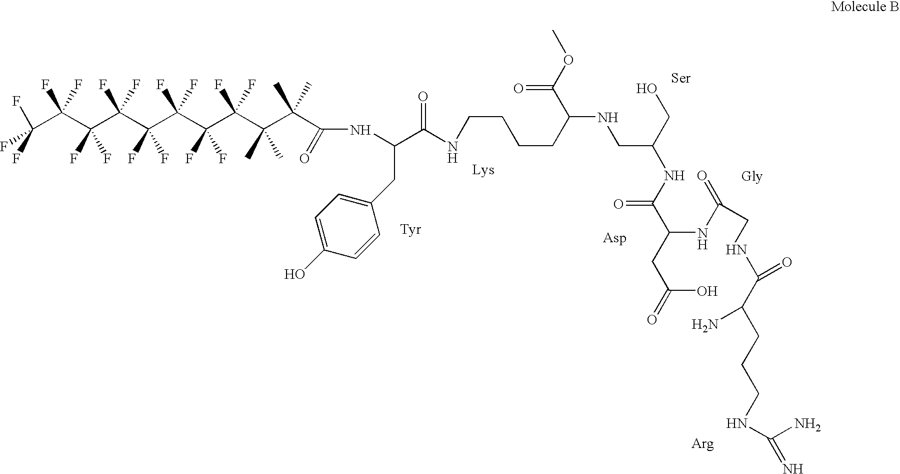

22. The compound as claimed in claim 1, corresponding to formula (Ic): 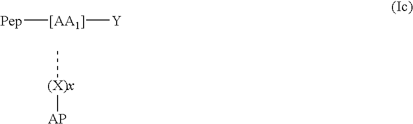
in which:
AP represents an active principle capable of acting on a biological target;
Pep represents a peptide chain containing from 2 to 10 amino acids;
x represents an integer chosen from 0 and 1;
X represents a peptide chain comprising from 1 to 5 amino acids;
AA₁ represents an amino acid carrying an acid, amine, alcohol or thiol function on its side chain;
Y represents a fluorinated C₄-C₁₂ hydrocarbon-based chain containing a function chosen from 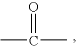
—NH, —O—CO—NH—, S and O that allows its attachment either to one of the ends of the amino acid AA₁, or to the side chain of AA₁.

23. The compound as claimed in claim 22, wherein one or more of the conditions below are verified:
Pep is a peptide recognized by αVβ3 integrins and AP is an antimitotic agent;
X, Pep or AA₁ contains at least one tyrosine residue;
X represents a chain of 1 to 3 amino acids.

24. The compound as claimed in claim 22 corresponding to one of formulae C, D and F:

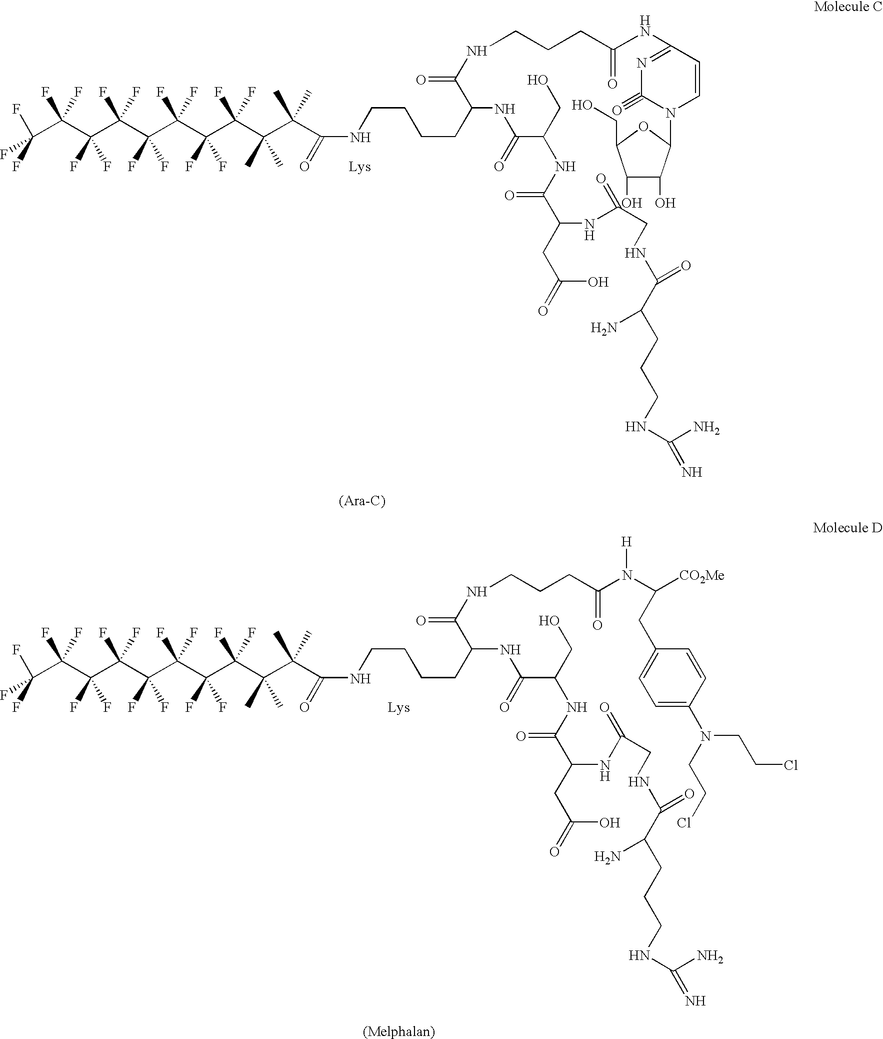

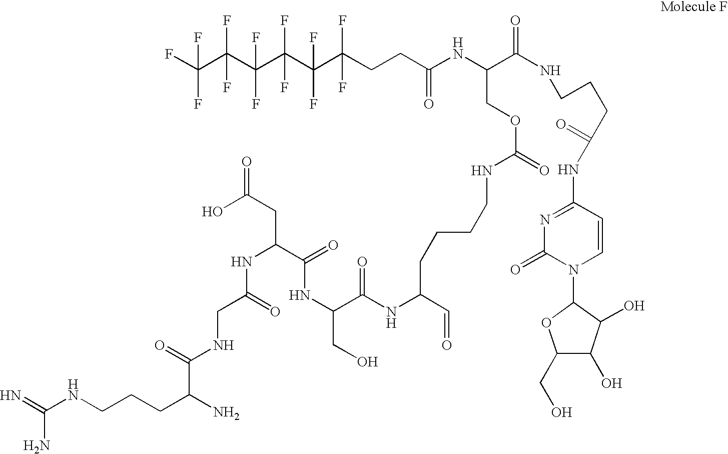

25. The compound as claimed in claim 22, wherein AP is adriamycin and X or Pep contain a Gly-Phe-Leu-Gly fragment.

26. The compound as claimed in claim 22, wherein AP is chosen from melphalan, 5-fluorouracil and imatinib mesylate.

27. A pharmaceutical composition comprising a compound as claimed in claim 1 in a pharmaceutically acceptable carrier.

28. The use of a compound of formula A, C, D or F as claimed in claim 16, for preparing a pharmaceutical composition intended to prevent and/or treat cancer.

29. The use of a compound of formula B as claimed in claim 21, for preparing a pharmaceutical composition intended to detect the presence of cancerous cells.

30. The use of a compound of formula E as claimed in claim 18, for preparing a pharmaceutical composition intended to prevent and/or treat pathologies associated with oxidative stress and with the formation of oxygenated free-radical species.
